(19) United States
(12) Patent Application Publication
(10) Pub. No.: US 2002/0058077 A1
(43) Pub. Date: May 16, 2002

(54) Cancer chemotherapeutical and chemopreventive agent

(75) Inventors: Han Ming Shen, Singapore (SG); Choon Nam Ong, Singapore (SG)

Correspondence Address: Attention: Mr. Richard J. Polley, KLARQUIST SPARKMAN CAMPBELL LEIGH & WHINSTON, LLP, One World Trade Center, 121 S.W. Salmon Street, Suite 1600, Portland, OR, 97204, US

(73) Assignee: National University of Singapore

(21) Appl. No.: 10/003,746

(22) Filed: Nov. 14, 2001

Related U.S. Application Data
(60) Provisional application No. 60/248,962, filed on Nov. 15, 2000.

(51) Int. Cl. A61K 35/78

(52) U.S. Cl. 424/764

(57) ABSTRACT

This invention relates to the use of parthenolide or derivative thereof and chrysanthemum ethanolic extract containing parthenolide in the treatment and prevention of cancer, including cancer associated with an increased COX-2 expression and increased constitutive activation of NF-κB.

### RELATED APPLICATION

This application claims the benefit of U.S. Provisional Application No. 60/248,962, filed Nov. 15, 2000.

### FIELD OF THE INVENTION

The present invention relates to the chemotherapeutic and chemopreventive effect of parthenolide and chrysanthemum ethanolic extract containing parthenolide.

### BACKGROUND OF THE INVENTION

Chrysanthemum has been documented for centuries by herbalists in Europe for the treatment of numerous ailments including fever, arthritis and migraine (Berry, 1984). In traditional Chinese medicine, chrysanthemum has been widely used as a herbal remedy for various disorders for more than two thousand years. Chrysanthemum is rich in sesquiterpene lactones (SL) and parthenolide is one of the major components in chrysanthemum extract with the highest concentration in the extract of Chrysanthemum parthenium/Tanacetum parthenium.

Parthenolide, as shown in FIG. 1, contains an α-methylene-γ-lactone ring and an epoxide which are able to interact readily with nucleophilic sites of biological molecules. Parthenolide possesses remarkable anti-inflammatory property and one of the important mechanisms is related to its inhibitory effect on arachidonic acid metabolism and prostaglandin (PG) production through its direct interaction with cyclooxygenase (COX) enzyme (Capasso, 1986; Pugh and Sambo, 1988; Sumner et al., 1992) or suppression of COX-2 expression via the inhibition of protein tyrosine kinase in lipopolysaccharide (LPS)-stimulated macrophages (Hwang et al., 1996). Parthenolide has also been shown to be a potent nuclear factor-κB (NF-κB) inhibitor as it can specifically suppress the activity of the IKK complex and the subsequent degradation of the NF-κB inhibitory proteins (IκBα and IκBβ) (Bork et al., 1997; Hehner et al., 1998; 1999). COX-2 is one of the target genes regulated by NF-κB (Appleby et al., 1994; Tazawa et al., 1994; Yamamoto et al., 1995), but it is not known whether parthenolide is capable of inhibiting COX expression and PG production by its effect on NF-κB or whether parthenolide is capable of inhibiting COX expression in cancer cells.

NF-κB is a ubiquitous nuclear transcription factor that governs the expression of various important genes which are closely related to a number of physiological and pathological processes including inflammation, development, immunity and cancer (Chen et al., 1999; Grossmann et al., 1999). In most cell types, NF-κB resides in the cytoplasm in an inactive form bound to an inhibitory protein known as IκB (Karin, 1999). Upon activation, IKB is phopsphoylated by an upstream kinase (IKK) and eventually proteolytically degraded, which leads to the translocation of NF-κB into the nucleus (Karin and Ben-Neriah, 2000; Israel, 2000). In nuclei, it then binds to a specific binding site located in the promoter or enhancer region of various target genes. Up to date, there are more than 150 genes found to be regulated by NF-κB (Pahl, 1999).

One of the key mechanisms involved in the biological functions of NF-κB is related to its regulatory effects on apoptosis (Barkett and Gilmore, 1999). Although whether NF-κB promotes or inhibits apoptosis appears to depend on the specific cell type and the nature of stimuli, under most circumstances, NF-κB acts as an apoptosis blocker, especially in TNF-α-induced apoptotic cell death (Barkett and Gilmore, 1999; Aggarwal, 2000). Such finding well explains the results that in most cell types TNF-α is not cytotoxic unless the cells are simultaneously treated with RNA or protein synthesis inhibitors which blocks the expression of NF-κB dependent anti-apoptotic genes (Baichwal and Baeuerle, 1997). The list of such anti-apoptotic genes includes Bcl-2 family proteins, inhibitors of apoptosis proteins, Mn-superoxide dismutase and COX-2 (Barkett and Gilmore, 1999).

COX or prostaglandin H synthase is the enzyme that catalyzes rate-limiting steps in the biosynthesis of prostaglandins (PGs). In contrast to COX-1, the constitutive form that plays an important role in cell homeostasis, COX-2 is the inducible form and mainly involved in the onset of inflammation and mitogenic responses (Dubois et al., 1998; Williams et al., 1999). Since the identification and cloning of COX-2 (Kujubu et al., 1991), accumulating evidence from epidemiological investigations, clinical trials, animal models, and various in vitro experiments supports the critical role of COX-2 in carcinogenesis (Prescott and Fitzpatrick, 2000; Williams et al., 1999; Dubois et al., 1998; Taketo, 1998a; 1998b). For instance, upregulation of COX-2 expression and PG production are commonly found in many cancer cells such as colorectal cancer and a number of COX-2 inhibitors such as nonsteroidal anti-inflammatory drugs (NSAIDs) are able to selectively induce apoptotic cell death in cancer cells (Sano et al., 1995; Shiff et al., 1995; Kutchera et al., 1996; Sheng et al., 1997; Chinery et al., 1998). Most probably, COX-2 promotes cell proliferation and inhibits apoptosis in cancer cells through a dual-mechanism: (i) enhanced synthesis of PGs, which favour the growth of malignant cells by increasing cell proliferation (Sheng et al., 1997; 1998), and (ii) reduced level of arachidonic acid, which has recently been found to promote apoptosis in cancer cells (Chan et al., 1998; Cao et al., 2000).

Several preliminary studies have shown that parthenolide is capable of inhibiting DNA synthesis and cell proliferation in a number of cancer cells, but the mechanisum of action involved is not known (Woynarowski and Konopa, 1981; Hall et al., 1988; Ross et al., 1999). A more recent study demonstrated that parthenolide is capable of increasing the sensitivity of human breast cancer cells to paclitaxel, a chemotherapeutical drug (Patel et al., 2000). However, there is no direct evidence and it is presently not known if parthenolide itself is effective to treat or prevent cancer. Similarly, chrysanthemum has not been used as a remedy to prevent or treat cancer.

### SUMMARY OF THE INVENTION

The invention provides a method of preventing or treating cancer comprising administering to an animal in need of such prevention or treatment an effective amount of Chrysanthemum ethanolic extract, parthenolide or a derivative thereof.

In one embodiment, the animal is human patient and the cancer is associated with an increased expression of COX-2. In another embodiment, the cancer is associated with an increased constitutive activation of NF-κB. Yet in another embodiment, the cancer is colorectal cancer, nasopharyngeal cancer, prostate cancer, bladder cancer, brain tumour, breast cancer, or skin cancer.

The invention also provides a kit comprising chrysanthemum ethanolic extract, parthenolide, or a derivative thereof and instructions for use in the treatment of cancer or prevention of cancer, including, colorectal cancer, nasopharyngeal cancer, prostate cancer, bladder cancer, brain tumour, breast cancer or skin cancer.

A composition comprising parthenolide or derivative thereof and a pharmaceutically acceptable diluent or carrier is also provided.

Other features and advantages of the present invention will become apparent from the following detailed description. It should be understood, however, that the detailed description and the specific examples, while indicating preferred embodiments of the invention are given by way of illustration only, since various modifications within the spirit and scope of the invention will become apparent to those skilled in the art from this detailed description. Such modifications are also intended to be within the scope of the invention.

### BRIEF DESCRIPTION OF THE DRAWINGS

FIG. 1 is the chemical structure of parthenolide.

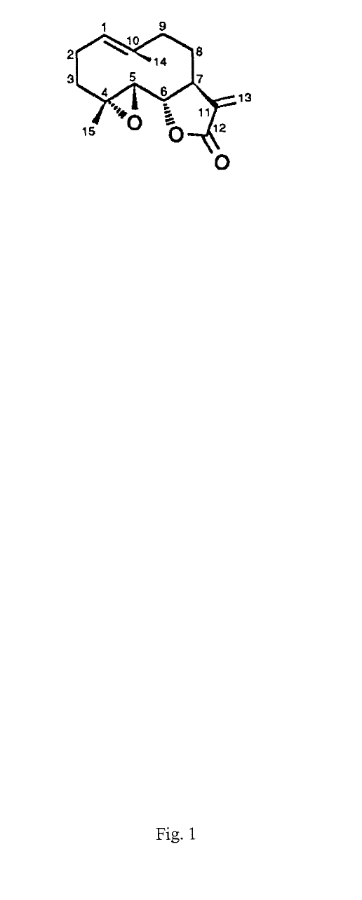

FIG. 2 shows the level of COX-2 protein in CNE1 and CNE2 cells and the cytotoxic effect of parthenolide on these cells. (A) shows detection of COX-1 and COX-2 level in both CNE1 and CNE2 cells using western blot. Unstimulated control cells were cultured in RPMI 1640 medium without FBS for 24 h before cells were scraped for the collection of whole cell and cytosolic extracts, as described in Example 2—Experimental Procedures. The doublet of COX-1 most likely represents differentially glycosylated forms of COX-1 protein (Jobin et al., 1998). (B) is a measurement of parthenolide cytotoxicity by the percentage of LDH leakage. After CNE1 and CNE2 cells were treated with parthenolide (PN, ranging from 5 to 100 μM) for 24 h, a fraction of culture medium was collected for the measurement of LDH activity. Data are presented as mean ±SD from 3 independent experiments. ** p<0.01 between CNE1 and CNE2 cells under the same parthenolide concentration (Student's t test).

FIG. 3 shows parthenolide induced apoptosis in TNF-α treated CNE1 cells. Cells were pretreated with parthenolide (25 μM×4 h), PDTC (25 μM×30 min), Act D (5 μg/ml×30 min), CHX (5 μg/ml×30 min), or NS398 (10 μM×30 min) prior to TNF-α exposure (15 ng/ml×24 h). Apoptosis was detected by cell morphological changes (A), PARP cleavage (B) and TUNEL assay (C). In A and B: a—control cells, b—TNF-α only, C—parthenolide only, d—arthenolide+TNF-α, e—PDTC+TNF-α, f—Act D+TNF-α, g—CHX+TNF-α, and h—NS398+TNF-α. In C: TUNEL assay was carried out in both attached and detached cells and analysed using flow cytometry and confocal microscopy.

FIG. 4 is quantification of parthenolide-induced apoptosis in TNF-α treated CNE1 cells. (A) shows dose-dependent increase of cell detachment caused by parthenolide in CNE1 cells. Cells were pretreated with various concentrations of parthenolide for 4 h before TNF-α exposure (15 ng/ml×24 h). Data are presented as mean ±SD from 3 independent experiments. * p<0.05 and ** p<0.01 compared to the control group (one-way ANOVA with Scheffe's test). In (B), cells were pretreated with parthenolide (25 μM×4 h), PDTC (25 μM×30 min), Act D (5 μg/ml×30 min), CHX (5 μg/ml×30 min), or NS398 (10 μM×30 min) prior to TNF-α exposure (15 ng/ml×24 h). Data are presented as mean ±SD from 3 independent experiments. * p<0.05 and ** p<0.01 compared between cells with or without TNF-α exposure (Student's t test).

FIG. 5 shows inhibtion by parthenolide of TNF-α induced NF-κB activation as determined by EMSA. In (A), CNE1 cells were first treated with parthenolide (25 μM×4 h) and then exposed to TNF (15 ng/ml) for indicated period of time (from 5 min to 6 h). In (B), cells were pretreated with PDTC (25 μM) for 30 min before TNF-α exposure (15 ng/ml×1 h). (C) is competition and supershift assay: lane 1-control cells, lane 2- TNF-α (15 ng/ml×30 min), lane 3-parthenolide only (25 μM×4.5 h), lane 4—nuclear protein as in lane 2 incubated with 50-fold excess amount of unlabeled cold NF-κB probe, lane 5- nuclear protein as in lane 2 incubated with 50-fold excess amount of unlabeled cold AP-1 probe, and lane 6-supershift with anti-p65 antibody. The preparation of nuclear extract and EMSA were carried out as described in detail in Example 2—Experimental Procedures.

FIG. 6 shows inhibitory effects of parthenolide on NF-κB activation is pre-treatment time and dose dependent. In (A), cells were pretreated with parthenolide (25 μM) from 0 to 4 h prior to TNF-α exposure (15 ng/ml) for 30 min. In (B), cells were pretreated with various concentrations of parthenolide (from 5 to 25 μM) for 4 h before TNF-α exposure (15 ng/ml×30 min). In lane 3, the concentration of PN used was 25 μM. At the end of treatment, cells were collected and both the nuclear and cytosolic extracts were prepared. Cytosolic IκBα degradation, nuclear p65 translocation and NF-κB-DNA binding were then detected using western blot and EMSA, respectively.

FIG. 7 shows inhibtion by parthenolide of IκBα nuclear localization in TNF-α treated CNE1 cells. (A) shows changes in IκBα in both cytosolic and nuclear extracts, and (B) shows changes in p65 in both cytosolic and nuclear extracts. The reaction was terminated after cells were pretreated with parthenolide (25 μM×4 h), followed by TNF-α exposure (15 ng/ml×30 min). The content of β-actin was also determined as a loading control.

FIG. 8 shows changes in the transcriptional activity of NF-κB as determined by luciferase reporter gene assay. In (A), cells were first transiently transfected with pNF-κB-luc vector for 24 h. Pretreatments with parthenolide (25 μM×4 h), PDTC (25 μM×30 min), Act D (5 μg/ml×30 min), or CHX (5 μg/ml×30 min) were conducted prior to TNF-α exposure (15 ng/ml×24 h). In (B), cells were pretreated with different doses of parthenolide for 4 h, followed by TNF-α exposure (15 ng/ml×24 h). After treatments, the luciferase activity was measured in the cellular extracts using a luciferase assay kit. Data are presented as mean ±SD (n=3). ** p<0.01 compared to the control group treated with TNF-α only (one-way ANOVA with Scheffe's test).

FIG. 9 shows direct interference by parthenolide with the DNA binding activity of NF-κB. Nuclear extracts from TNF-α-treated cells containing activated NF-κB were pooled and incubated with various concentrations of parthenolide (0 to 25 μM) for 1 h at room temperature. The control group was treated with the 0.1% of DMSO which is the same as that used in the highest concentration of parthenolide (25 μM). The incubated extracts were then analysed by EMSA.

FIG. 10 is RT-PCR analysis of mRNA expression of COX-2 in parthenolide-pretreated CNE1 cells. In (A), cells were first pretreated with parthenolide (25 μM×4 h), followed by TNF-α exposure (15 ng/ml) for indicated periods of time. In (B), cells were either pretreated with various concentrations of parthenolide for 4 h or Act D (5 μg/ml) for 30 min, followed by TNF-α exposure (15 ng/ml) for 6 h. In lane 3, the concentration of parthenolide used was 25 μM. The mRNA level of G3PDH was also determined as a control.

FIG. 11 shows reduction of COX-2 protein level in parthenolide-pretreated CNE1 cells. In (A), cells were first pretreated with parthenolide (25 μM×4 h), followed by TNF-α exposure (15 ng/ml) for 12 h or 24 h. In (B), cells were either pretreated with various concentrations of parthenolide for 4 h or CHX (5 μg/ml) for 30 min, followed by TNF-α exposure (15 ng/ml) for 24 h. In lane 3, the concentration of parthenolide used was 25 μM. The protein level of β-actin was also determined as a loading control.

FIG. 12 shows inhibition by parthenolide of PGE₂ production in TNF-α treated CNE1 cells. The level of PGE₂ in cell culture medium was determined using an EIA kit. Following the pre-treatment with either parthenolide (4 h at the different concentrations indicated) or NS398 (10 μM×30 min), CNE1 cells were exposed to TNF-α (15 ng/ml) for 24 h. The results are presented as folds over the control group (mean ±SD from at least 3 independent experiments). ** p<0.01 compared to the control group treated with TNF-α-only (one-way ANOVA with Scheffe's test).

FIG. 13 shows cell growth inhibition by parthenolide in both HCA-7 and HCT-116 cells in vitro. In (A), COX-1 and COX-2 level in both cells was determined by western blot. In (B), the level of PGE₂ production in two cell lines was determined by EIA. (C) shows cell growth inhibition measured by MTT assay. Data are presented as mean ±SD (n=8). ** p<0.01 compared to HCA-7 cells at the same parthenolide concentration (Student's t test).

FIG. 14 shows parthenolide-induced apoptosis in both HCA-7 and HCT-116 cells determined by DNA content/cell cycle analysis. The percentage of sub-G1 cells was indicated in each histogram and marked as M1. Cells were treated with various concentrations of parthenolide for 24 h. Data are one representative set from three independent experiments.

FIG. 15 shows cell growth inhibition by parthenolide in HCA-7 cells in vivo. (A) shows changes of body weight throughout the period of experiment, (B) shows changes of tumor weight and (C) shows changes of tumor volume. Both the tumor weight and volume were measured at the time of animal sacrifice. Data are presented as mean ±SD (n=8). * p<0.05 and ** p<0.01 compared to the control group (Student's test).

FIG. 16 shows changes in BrdU incorporation in xenograft tumors. (A) represent microscopic images of BrdU-positive cells in HCA-7 xenografts (×400 magnification). Arrows indicate some typical BrdU-positive cells. The slide was stained using the BrdU labeling and detection kit (Roche) and counterstained with hematoxylin. (B) is quantification of BrdU incorporation expressed by the number of BrdU-positive cells in each field (×400 magnification). Total of 10 representative fields were selected and counted for each animal. Data are presented as mean ±SD (n=3). * p<0.05 compared to the control group (Student's test).

FIG. 17 shows induction of apoptotic cell death in xenograft tumors determined by TUNEL assay. (A) represent microscopic images of TUNEL-positive cells in HCA-7 xenografts (×400 magnification). Arrows indicate some typical TUNEL-positive cells. The slide was stained using the In Situ Cell Death Detection Kit (Roche) and counterstained with hematoxylin. (B) is quantification of apoptosis expressed by the number of TUNEL-positive cells in each field (×400 magnification). Total of 10 representative fields were selected and counted for each animal. Data are presented as mean ±SD (n—5). * p<0.05 and ** p<0.01 compared to the control group (Student's test).

### DETAILED DESCRIPTION OF THE INVENTION

The present invention relates to the novel finding that cancer cells are susceptible to the cytotoxic effect of parthenolide. The invention also relates to the novel finding that this cytotoxicity is mediated by apoptosis of cancer cells and further that parthenolide induces apoptosis by inhibiting NF-κB activation. The invention further relates to the finding that COX-2, whose expression is regulated by NF-κB is a molecular target of parthenolide and that parthenolide can induce apototic cell death by inhibiting of COX-2 expression as a consequence of inhibition of NF-κB activation.

More specifically, the inventors have found that human nasopharyngeal cancer cells and human colorectal cancer cells with an increased expression of COX-2 are more susceptible to the cytotoxic effect of parthenolide and that parthenolide at non-toxic concentrations induces apoptosis in these cells and sensitizes these cells to apoptosis on TNF-alpha treatment, and inhibits COX-2 expression and PGE₂ production in a dose-dependent manner.

The inventors have determined that in these cancer cells, COX-2 expression is induced following NF-κB activation. The inventors have also found that parthenolide treatment leads to inhibition of NF-κB activation. As such, it is believed that parthenolide inhibits NF-κB activation and prevents NF-κB DNA binding and transcription of target genes leading to inhibition of target gene expression, and that parthenolide inhibits the expression of COX-2 which is regulated by NF-κB, by inhibition of NF-κB activation.

There is growing evidence that COX-2 plays a critical role in carcinogenesis, and the inventors have shown that parthenolide, by inhibiting COX-2 expression, induces apoptosis in cancer cells. Moreover, the inventors have shown that parthenolide induced apoptosis is mediated by inhibition of NF-κB activation and further that parthenolide is more effective than NS398, a known inhibitor of COX-2 in inducing apoptosis. Parthenolide is therefore expected to be effective in inhibiting other anti-apoptotic genes regulated by NF-κB, by inhibiting NF-κB activation. Therefore, the invention provides a novel agent against cancers, including cancers associated with an increased expression of COX-2 or an increased constitutive activation of NF-κB.

The inventors have also shown that at non-toxic concentration, parthenolide is effective in inhibiting cancer cell proliferation in vivo. Therefore, parthenolide and chrysanthemum ethanolic extract containing parthenolide as a major component can be administered as a chemotherapeutic and chemopreventive agent against cancer, including cancers associated with an increased expression of COX-2, such as colorectal cancer, nasopharyngeal cancer, prostate cancer, bladder cancer, brain tumour, breast cancer, and skin cancer, and cancers associated with an increased constitutive activation of NF-κB, which include breast cancer, prostate cancer and colorectal cancer. Cancer associated with an increased constitutive activation of NF-κB may be readily determined by tests known to detect NF-κB activation. Normal tissues and cells do not generally express a detectable level of COX-2 as measured by conventional assays and it will be understood that “increased expression of COX-2” refers to a detectable increase in the level of COX-2 expression as compared to normal tissues and cells. Similarly, it will be understood that “increased constitutive activation of NF-κB” refers to a detectable increase in the level of activated NF-κB as compared to the constitutive level of activated NF-κB in normal tissues and cells.

Chrysanthemum ethanolic extract and parthenolide therefore may be administered to treat any animal, including a human patient suffering from cancer. They may also be administered to an animal, including human patients who have an increased risk of cancer, for example due to family history, or environmental risk factors, as a preventive measure. For example, in these patients, the level of COX-2 expression or activated NF-κB may be monitored and parthenolide or chrysanthemum ethanolic extract may be administered to prevent the onset of cancer. Parthenolide is a major component of chrysanthemum extract which has been widely used as a herbal remedy for centuries. Parthenolide and chrysanthemum ethanolic extract therefore provides a dietary approach, which may be ideally suited for prevention of cancer. Patients receiving treatment may be monitored for the effectiveness of the treatment in the known manner, including for example, in the case of patients receiving treatment to prevent or treat cancer associated with an increased expression of COX-2, by monitoring the level of expression of COX-2 in cell or tissue samples. The level of activated NF-κB may also be monitored to assess the effectiveness of the treatment.

An effective amount refers to that amount effective, at dosages and for a period of time necessary to achieve the desired therapeutic result, which may include the amount at which any toxic or detrimental effects are outweighed by therapeutically beneficial effects. The effective amount will vary according to various factors and may be readily determined by those skilled in the art. For example, the optimal daily dose of chrysanthemum extract and parthenolide may be readily determined by methods known in the art and may depend on the type of cancer, the condition of the patient being treated, the therapeutic response, and whether the patient is receiving other chemotherapeutical or chemopreventive agents. As discussed in more detail in the Examples, parthenolide exhibits 50% effectiveness concentration of about 23 μM, similar to NS398, a known specific COX-2 inhibitor. It is therefore expected that the optimal daily dose of parthenolide will be similar to known NSAID's. In one embodiment therefore, about 200 to 800 mg of parthenolide can be administered daily in a single or multiple dosage regimen. The amount of parthenolide present in chrysanthemum extract may be readily determined by known methods and an amount of chrysanthemum extract containing about 200 to 800 mg of parthenolide may also be administered daily in a single or multiple dosage regimen.

As the anti-carcinogenic property of parthenolide is largely due to the α-methylene-γ-lactone group present in the compound, it will be understood that derivatives of parthenolide which retain this lactone structure can have the same anti-carcinogenic effect and are also within the scope of the invention. For example, the α-methylene-γ-lactone group can be linked to a cyclohexadienone structure to enhance effectiveness. The term “derivative of parthenolide” is intended to encompass not only any such modified parthenolide but other structurally similar compounds which have a α-methylene-γ-lactone group and which can therefore be expected to have similar properties to parthenolide. In one embodiment, such compounds may offer conjugation sites for ester or other moiety.

Chrysanthemum ethanolic extract and parthenolide or a derivative thereof will most typically be administered orally, for example, with an inert diluent or with an assimilable edible carrier, or enclosed in hard or soft shell gelatin capsules, or compressed as tablets, or incorporated directly in food. However, appropriate route of administration may vary depending on the cancer to be prevented or treated. For example, chrysanthemum ethanolic extract, parthenolide or derivative thereof may be administered topically as a cream, gel or transdermal patch to prevent or treat skin cancer. To prevent or treat nasopharyngeal cancer, chrysanthemum ethanolic extract, parthenolide or derivative thereof may be adminstered intra-nasally using a nasal spray. Parenteral administration may also be suitable and include intravenous, intraperitoneal, subcutaneous, intramuscular, transepithelial, nasal, intrapulmonary, intrathecal, and rectal modes of administration. Parenteral administration may be by continuous infusion over a selected period of time. Suitable pharmaceutically acceptable carriers and diluents known in the art may therefore be combined in the preparation of suitable dosage forms and chrysanthemum ethanolic extract, parthenolide or derivative thereof may be administered alone or in combination with any such pharmaceutically acceptable carriers or diluents.

Compositions comprising chrysanthemum extract or parthenolide or derivative thereof and any such diluent or carrier are also within the scope of the present invention and can be prepared by known methods by combining an effective amount of the active substance in a mixture with a pharmaceutically acceptable diluent or carrier.

For oral therapeutic administration, chrysanthemum ethanolic extract or parthenolide or derivative thereof may be incorporated with one or more suitable excipients and used in the form of ingestible tablets, buccal tablets, troches, capsules, elixirs, suspensions, syrups, wafers and the like. The pharmaceutical forms suitable for parenteral use include sterile aqueous solutions or dispersion and sterile powders for the extemporaneous preparation of sterile injectable solutions or dispersions. Solutions can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (1990-18^{th} edition) and in The United States Pharnacopeia: The National Formulary (USP 24 NF19) published in 1999.

Chrysanthemum ethanolic extract, parthenolide or derivative thereof may also be packaged as a kit which includes instructions for use in the treatment or prevention of cancer. A kit comprising chrysanthemum ethanolic extract, parthenolide or derivative and any such instructions are therefore also within the scope of the invention.

All references cited herein are fully incorporated by reference.

The present invention is further described in the examples below. The examples are illustrative only and are not intended to limit the scope of the invention

### EXAMPLES

### Example 1

### Chrysanthemum Ethanolic Extract

Extraction of Parthenolide.

Crude extract of chrysanthemum can be prepared by boiling dried chrysanthemum flower in water with stirring for 20 minutes. Extraction using this method generally gives very low yield. Less than 0.15% of pure parthenolide can be obtained, based on dry weight of the flower. Addition of ethanol (90:10, solvent: water, v/v) improved the yield to about 0.8%. No significant differences in terms of yield and recovery were observed when using either bottle-stirring or Soxhlet. For 20 gm of dried chrysanthemum about 160-180mg of parthenolide can be obtained if methanol is used. The purity can be confirmed by using HPLC with a photodiode array detector using pure parthenolide. The term “chrysanthemum ethanolic extract” as used herein is intended refer to any extract in which the yield of parthenolide has been improved from the water extract and to distinguish any such extract from the chrysanthemum water extract.

### Example 2

### Experimental Procedures

Materials

The following chemicals or reagents were purchased from Sigma (St. Louis, Mo.): parthenolide, TNF-α, pyrrolidinedithiocarbamate (PDTC), actinomycin D (Act D), cycloheximide (CHX), bovine serum albumin (BSA), poly(dI-dC), ammonium persufate, phenylmethylsulfonyl fluoride (PMSF), aprotinin, leupeptin, penicillin and streptomycin. NS398 (a specific COX-2 enzyme inhibitor) and Nonidet P-40 were from Calbiochem (La Jolla, Calif.). RPMI-1640 medium, T4 polynucleotide kinase and the forward buffer, TRIZOL RNA extraction reagent, Superscript II reverse transcriptase were from Life Technologies (Gaithersburg, Md.). 40% Acrylamide, 2% Bis-acrylamide, TEMED, and Rainbow™ protein marker were all from Amersham Pharmacia (Piscataway, N.J.). Foetal bovine serum (FBS) was from Hyclone (Logan, Utah). NF-κB (p65) monoclonal antibody, IκBα monoclonal antibody, COX-1 polyclonal antibody were all from Santa Cruz (Santa Cruz, Calif.). Anti-COX-2 monoclonal antibody was from BD Transduction Laboratories (Los Angeles, Calif.). The secondary antibodies (horseradish peroxidase conjugated goat anti-mouse IgG and rabbit anti-goat IgG), and the enhanced chemiluminescence substrate were from Pierce (Rockford, Ill.). NF-κB and AP-1 consensus oligonucleotides, TransFast™ transfection reagent, recombinant RNase inhibitor, and 100 bp DNA marker were all from Promega (Madison, Wis.). DyNAzyme II DNA polymerase was purchased from Finnzymes (Espoo, FI). Oligo d(T) primer and dNTP were from New England Biolabs (Beverly, Mass.). The Mercury Pathway Profiling System containing both the pNF-κB-luc and pTAL-luc vectors were obtained from Clontech (Polo Alto, Calif.). γ-p32 ATP was from NEN Life Science (Boston, Mass.). SDS ready gel, Laemmli sample buffer and the protein quantification kit were purchased from Bio-Rad (Hercules, Calif.). The PGE₂ enzyme immunoassay kit was purchased from Cayman (Ann Arbor, Mich.). TdT-mediated dUTP nick end labeling (TUNEL) assay kit (In Situ Cell Death Detection Kit) was from Roche (Mannheim, Germany).

Cell Culture and Treatment of CNE 1 and CNE 2 Cells

The human nasopharyngeal cancer (NPC) CNE1 and CNE2 cells were obtained from Sun Yet-sat University of Medical Sciences (Guangzhou, China) and cultured in RPMI-1640 medium supplemented with 10% FBS and 100 units/ml penicillin and 100 μg/ml streptomycin. TNF-α (final concentration 15 ng/ml) was used as the positive stimulus to promote NF-κB activation in cultured cells. The stock solution of parthenolide (100 mM) was prepared in DMSO and cells were pretreated with various concentration of parthenolide (5 to 25 μM) for up to 4 h prior to TNF-α exposure. The DMSO concentration was always lower than 0.025% in treated cells, and the control group was balanced with the same concentration of DMSO. PDTC (25 μM), Act D (5 μg/ml), CHX (5 μg/ml), and NS398 (10 μM) were added into cell culture medium 0.5 h before TNF-α exposure. The treatments were terminated at designated time points for various experiments, as described in details in the Results. In some experiments, parthenolide was added into the culture medium 12 h after TNF-α exposure, for the purpose of evaluating the direct interference of parthenolide with COX-2 enzyme activity.

Cell Culture and Treatment of HCT-7 and HCA-116 Cells

The human colorectal adenocarcinoma cell line HCA-7 colony 29 was kindly provided by Dr. Susan C. Kirkland (University of London, UK). HCT-116 cells were purchased from ATCC (Rockville, Md.). Both cell lines were cultured in McCoy's 5A medium (Sigma, St Louis, Mo.) supplemented with 10% FBS, 100 units/ml penicillin, 100 μg/ml streptomycin, pH 7.4 at 37° C. in 5% CO₂. Parthenolide (ordered from Biomol, Plymouth Meeting, Pa.) and Celecoxib (provided by Pharmacia, St Louis, Mo.) were dissolved in DMSO (100 mM) as stock and further diluted with FBS-free medium to desired concentrations. Control groups received same concentration of DMSO. After designated treatments in FBS-free medium, cells were collected for various analysis.

Preparation of Cytosolic, Nuclear and Whole Cell Extracts

Both the nuclear and cytosolic protein extracts were prepared according to published methods with modifications (Hehner et al., 1998; Gallois et al., 1998). After designated treatments, cells were collected using cell scrapper and washed with cold PBS twice. Cells (about 3-4×10⁶) were then resuspended in ice-cold 150 μl Buffer A (10 mM HEPES, pH 7.9, 1.5 mM MgCl₂, 10 mM KCl, 0.5 mM DTT, 0.5 mM PMSF, 1 μg/ml leupeptin, and 1 μg/ml aprotinin). After incubation on ice for 15 min, Nonidet P-40 (final concentration 0.3%) was added into the cell suspension and mixed gently. The cytosolic extracts were collected after cells were centrifuged at 2,000× g for 10 min at 4° C. The nuclei pellets were then resuspended in 40 μl of Buffer B (20 mM HEPES, pH 7.9, 1.5 mM MgCl₂, 450 mM NaCl, 25% glycerol, 0.2 mM EDTA, 0.5 mM DTT, 0.5 mM PMSF, 1 μg/ml leupeptin, 1 μg/ml aprotinin), and incubated on ice for 30 min with gentle votex once every 5 min. The nuclear extracts were collected after centrifugation (20,000× g for 15 min, 4° C.). For the preparation of the whole cell extracts, cells were resuspended in a cell lysis buffer (Buffer C, 50 mM Tris-HCl, pH 8.0, 150 mM EDTA, 1% Triton X-100, 0.5% SDS, 1 mM PMSF, 1 ∥g/ml aprotinin and 1 μg/ml leupeptin), and lysed on ice for 30 min, followed by centrifugation (10,000× g for 20 min, 4° C.) for the collection of supernatant. Protein concentration was quantified using a Bio-Rad protein assay kit and all samples were stored at −80° C. after dilution using respective buffers to 1 μg/μl.

Electrophoretic Mobility Shift Assay (EMSA)

The DNA binding activity of the nuclear protein was tested according to established method with modifications (Hehner et al., 1998). NF-κB consensus oligonucleotides (5′-AGTTGAGGGGACTTTCCCAGGC-3′ and 3′-TCAACTCCCCTGAAAGGGTCCG-5′) were labelled with p32 using T4 kinase and purified through a G50 column. Equal amounts of nuclear protein (5 μg) were incubated with 100,000 cpm labelled NF-κB oligonucleotides in 5× reaction buffer (Buffer D, 100 mM HEPES/KOH, pH 7.9, 20% Glycerol, 1 mM DTT, and 300 mM KCl) for 30 min at room temperature, in the presence of 2 μg poly(dI-dC) and 2 μg BSA in a total volume of 20 μl. In the competition experiments, 50-fold excess amount of specific unlabelled cold probe (NF-κB) or non-specific unlabelled cold probe (AP-1, 5′-CGCTTGATGAGTCGACCGGAA-3′ and 3′-GCGAACTACTCAGTCGGCCTT-5′) were incubated with the nuclear protein for 30 min prior to the addition of ³²P-labelled NF-κB oligonucleotides. In the supershift experiments, 0.2 μg of anti-p65 monoclonal antibody was added into the reaction mixture and incubated for 30 min on ice, followed by the addition of ³²P-labelled NF-κB oligonucleotides and incubation for another 30 min at room temperature. The DNA-protein complexes were resolved on a 5% polyacrylamide gel (Vertical gel electrophoresis apparatus (Gibco BRL Model v16-2) at 150 V for 1.5 h. Gels were then dried and exposed to an x-ray film (Kodak) at −80° C. overnight.

Western Blotting

Various cell extracts (cytosolic, nuclear or whole cell) were prepared as described earlier. Equal amounts (30 μg) of protein were separated on SDS-polyacrylamide gel in the Mini-PROTEAN II system (Bio-Rad). After electrophoresis, the protein was transferred onto a Hybond-C nitrocellulose membrane (Amersham) at 4° C. using the Mini Trans-Blot Module (Bio-Rad). The membrane was blocked overnight with 5% nonfat milk in TBST (10 mM Tris-HCl, pH 7.5, 100 mM NaCl, 0.1% Tween 20), and then incubated with various primary antibodies for 2 h at room temperature. After 5 washes with TBST, the membrane was exposed to respective secondary antibodies for 1 h. The blots were detected using the enhanced chemiluminescence method (Pierce).

Transient Transfection and Luciferase Reporter Gene Assay

The pNF-κB-luc vector and the negative control pTAL-luc vector were purchased as part of the Mercury Pathway Profiling Luciferase System from Clontech Laboratories. Another NF-κB luciferase reporter plasmid was kindly provided by Dr. Schmitz (German Cancer Research Center, Heidelberg, Germany). The propagation was conducted in competent cells JM109 using ampicillin as the selection marker. The plasmids were extracted and purified using Maxi-preps (Promega). The fragment sizes of each vector were confirmed using respective restriction enzyme digestions. The transient transfection of the above plasmids into cultured CNE1 cells were performed using TransFast™ Transfection Reagent (Promega) according to the manufacturer's protocol. Briefly, when cells reached about 80% confluence in 24-well plates (approximately 10×10⁴ cells per well in 0.5 ml culture medium), the growth medium from each well was removed by aspiration. The transfection mixture (200 μl in FBS-free RPMI 1640 medium) containing 0.5 μg DNA and 3 μl of the transfection reagent was added into each well. After incubated for 1 h at 37° C., 300 μl of prewarmed complete medium was added into each well and incubated for 24 h. Transfected cells were then subjected to various treatments. The luciferase activity was measured in the cellular extracts using a luciferase assay kit (Promega). Following the treatment, the cell lysate was collected from each well after the addition of 1× cell culture lysis reagent (50 μl/well, Promega). The relative light units (RLU) were then determined in a luminometer (Lumi-one, Trans Orchid, Tampa, Fla.) for a total period of 15 sec after a 5 sec delay time.

RNA Extraction and RT-PCR

RNA extraction was carried out using a total RNA extraction kit (TRIzol, Life Technologies). Briefly, cells were collected by scrapping after various treatments and washed with cold PBS once before they were lysed in the TRIzol reagent. After extracted with chloroform once, RNA was precipitated with isopropyl alcohol and washed with 75% ethanol. Finally the RNA samples were dissolved in DEPC-H₂O and quantified (1 A₂₆₀=40 μg/ml). Five micrograms of total RNA from each sample were subjected to reverse transcription using Superscript II reverse transcriptase (Life Technologies) in a total volume of 50 μl. For PCR, all the amplification reactions were carried out in 20 μl which included 200 pmol of each primers, 200 μM of each dNTPs, and 0.5 units of DyNAzyme II. The PCR was performed for 30 cycles, using a program of 95° C. for 30 sec, 57° C. for 30 sec, and 72° C. for 30 sec, followed by a 10 min extension at 72° C., in a Biometra T-gradient Thermal Cycler. The primers of human COX-2 were as follows: 5′-TTCAAATGAGATTGTGGGAAAATTGCT-3′ (sense) and 5′-AGATCATCTCTGCCTGAGTATCTT-3′ (anti-sense) (Hanif et al., 1996; Jobin et al., 1998). As an internal control, the level of glyceraldehydes-3-phosphate dehydrogenase (G3PDH) expression was also analyzed using the following primers: 5′-CCCTTCATTGACCTCAACTACATGG-3′ (sense) and 5′-CATGGTGGTGAAGACGCCAG-3′ (anti-sense) using the same condition as that of COX-2. The PCR conditions were optimised to achieve exponential amplification in which the PCR product formation is proportional to the starting cDNA. After PCR, products were size fractionated using 1.2% agarose gel, visualized by ethidium bromide staining and photographed.

Determination of PGE₂ Level Released from Cells—Enzyme Immunoassay (EIA)

The PGE₂ level in the cell culture medium after various treatments was determined using an EIA kit (Cayman) according to protocol from the manufacturer. Each sample was assayed at two dilutions and each dilution in triplets. The results were presented as the fold of increase comparing to that of the control group.

Cell Death and Apoptosis in CNE 1 Cells

Apoptosis was determined by the following assays: (i) cell morphological alterations examined under an inverted microscope, (ii) PARP cleavage determined by western blot, and (iii) TUNEL assay which was analysed using both flow cytometry and confocal microscope (Shen et al., 2000), in both attached and detached cells after various treatments. This assay detects DNA fragmentation, one of the hallmarks of apoptosis (Gorczyca et al., 1993). Based on the finding that almost all detached cells were apoptotic (TUNEL-positive), we used the percentage of detached cells as a quantitative parameter for apoptosis. In addition, for the measurement of cytotoxicity, the percentage of lactate dehydrogenase (LDH) leakage was also measured using an Abbott VP biochemical analyser with a test kit (Shen et al., 1995).

Cell Growth Inhibition and Induction of Apoptosis in HCA-7 and HCT-116 Cells

Parthenolide-induced inhibitory effect on cell proliferation in both HCA-7 and HCT-116 cells was determined using tetrazolium dye calorimetric test (MTT test) as established in our laboratory (Yang et al., 1999). The MTT absorbance was then read using a plate reader (Bio-Rad, model 3550) at 595 μm. Parthenolide-induced apoptotic cell death was determined by DNA content analysis and measurement of sub-G1 cells (Shen et al., 2000). It is well established that DNA fragmentation during apoptosis may lead to extensive loss of DNA content and result in a distinct sub-G1 peak when analyzed using flow cytometry (Nicoletti et al., 1991). Cells were first fixed and permeabilized with 70% ice cold ethanol for more than two hours, followed by incubation with the freshly prepared staining buffer (0.1% Triton X100 in PBS, 200 μg/ml RNase A, and 20 μg/ml PI) for 15 min at 37° C. Cell cycle was analyzed using flow cytometry with at least 10,000 cells for each sample. The histogram was abstracted using WinMDI 2.7 software and the percentage of cells in the sub-G1 phase was then calculated.

Nude Mice and Inoculation

Female Balb/c nude mice (5-6 weeks old) were purchased from the Animal Resources Centre (Murdoch, Australia) and maintained under SPF condition at the Animal Holding Unit, National University of Singapore. The animal experiments were conducted according to the University-approved guidelines. HCA-7 cells were cultured as described above. After cells reached about 80-90% confluence, they were trypsinzed and washed with PBS once. Cells were then resuspended in PBS at the concentration of 50×10⁶/ml. Each animal was injected with 0.1 ml of cell suspension (containing 5×10⁶ cells) at one side of the flank subcutaneously.

Treatment of Nude Mice

Three days prior to inoculation, animals were randomly divided into the following groups: (i) control (DMSO only), (ii) parthenolide low concentration (50 mg/kg body wt/day), (iii) parthenolide high concentration (150 mg/kg body wt/day), and (iv) Celecoxib as positive control (100 mg/kg body wt/day). All animals were fed with water and food ad libitum and the daily consumption of food and water was monitored throughout the study. Both parthenolide and Celecoxib were first dissolved in DMSO (1 g/ml) as stock and further diluted with vegetable oil. The drug-containing oil was then mixed with sterilized pellet diet (from Glen Frost Inc., Perth, Australia) and administered to the animals starting three days before inoculation.

The experiments were terminated and all animals were killed by cervical dislocation 4 weeks after inoculation. Both the tumor weight and tumor size were measured and the tumor volume was calculated according to the following formula: length×width×depth×0.52 (Goluboff et al., 1999). The tumor tissue was fixed in buffered formalin (10%) for paraffin sectioning.

BrdU Incorporation

The BrdU incorporation assay was used to measure DNA synthesis and cell proliferation. The test was conducted using a kit from Roche (Mannheim, Germany). One hour prior to killing, 3 mice from each group were injected intraperitoneally with undiluted BrdU labeling reagent (1.5 ml/100 g body wt). After the fixed tumor tissues were deparaffinized and rehydrolated, the immunohistochemical reaction was conducted following the manufacturer's protocol. The slides was counterstained with hematoxylin and mounted.

Evaluation of Apoptosis in Tissue

Apoptotic cell death in tumor tissues were quantified using an In Situ Cell Death Detection Kit (Roche, Mamiheim, Germany), also called TUNEL assay. The test was conducted according to the protocol from the manufacturer. After immunohistochemical staining, the slides were counterstained with hematoxylin and mounted.

Histological Analysis and Scoring

Slides were examined under light microscopy (×400 magnification) (Nikon Eclipse E600) by an experienced laboratory technician blind to grouping. For each tumor and each stain, 10 representative fields were counted. The results were expressed as the mean value of positive cells per field.

Statistics

All numerical data are presented as mean ±standard deviation (SD) from at least 3 independent experiments and analysed using Student's t test or one-way ANOVA with Scheffe's test. A p value <0.05 is considered statistically significant.

### Example 3

### Results

CNE1 Cells with Higher Expression Level of COX-2 are More Susceptible to Parthenolide Cytotoxicity

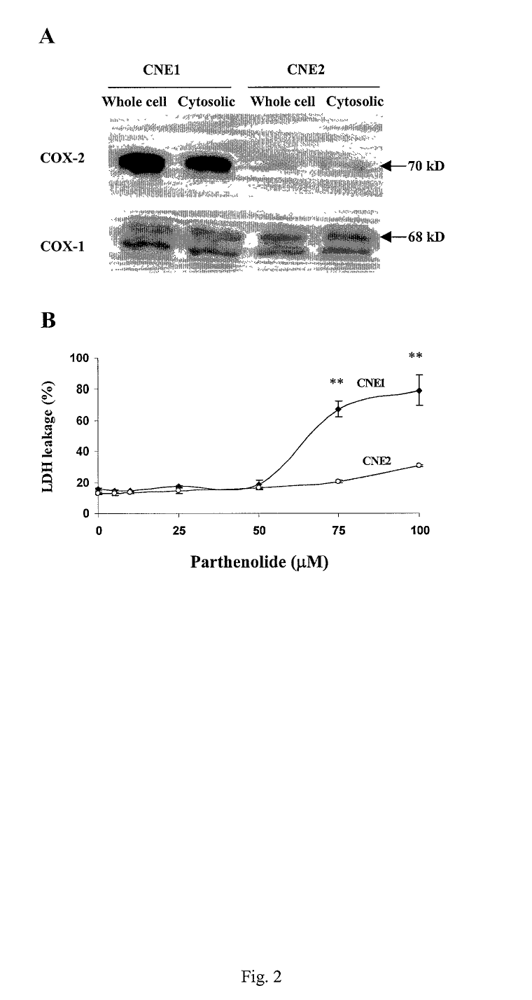

NPC is one of most common cancers in certain regions of Asia, while it is relatively rare in the West (Li et al., 1985). So far there is no report about the involvement of COX in this cancer. In the present study, we first tested the basal level of COX enzyme (both COX-1 and COX-2) in two NPC cell lines (CNE1 and CNE2 cells) which were originally established in China and widely used in the NPC study nowadays (Sizhong et al., 1983; Li et al., 1997). As shown in FIG. 2A, unstimulated CNE1 cells possess considerably high level of COX-2 protein, while in CNE2 cells the COX-2 protein is barely detectable. In contrast, both cells contain almost the same amount of constitutive COX-1. More interestingly, it is noted that CNE1 cells is much more susceptible to parthenolide toxicity at relatively higher concentrations (>50 μM) as determined by LDH leakage (FIG. 2B). Therefore, the close association between the COX-2 expression and parthenolide cytotoxicity indicates that COX-2 might be one of the molecular targets for the cytotoxic effect of parthenolide. In the following studies, we systematically examined the molecular mechanisms involved in the regulatory role of parthenolide on COX-2 expression and the causative role of such regulation in parthenolide cytotoxicity, using TNF-α as a positive stimulus in CNE1 cells.

Parthenolide Sensitises CNE1 Cells to Apoptosis on TNF-α Treatment

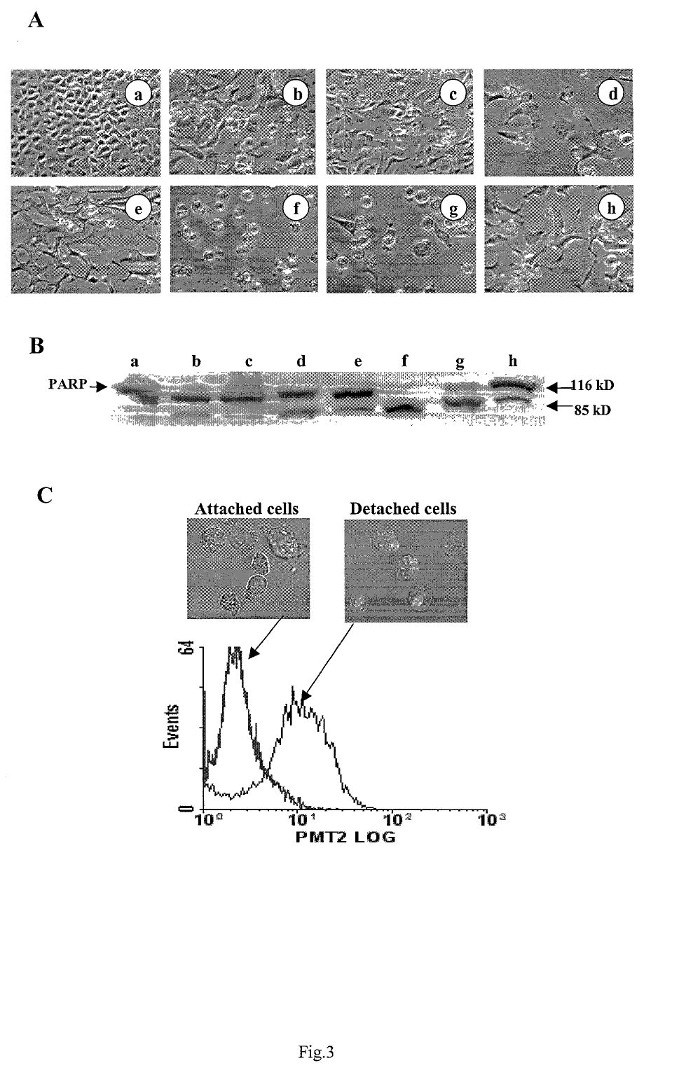

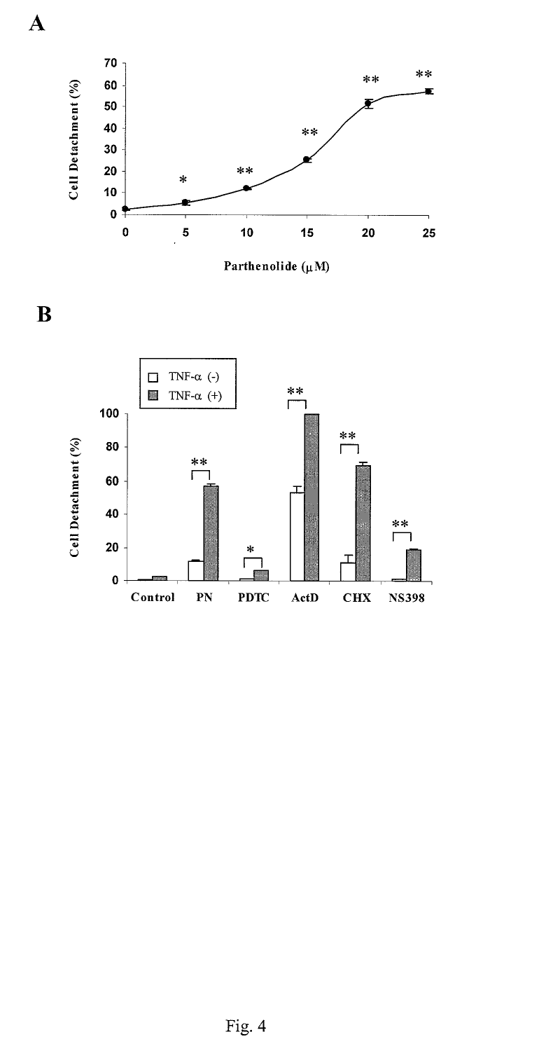

In this study, we used relatively low concentrations of parthenolide (from 5 to 25 μM) at which parthenolide itself is not cytotoxic to test the effect of parthenolide on TNF-α treated cells. Parthenolide alone induces apoptosis at higher concentrations as shown by in vivo study discussed below. The apoptotic cell death was examined by (i) morphological alterations, (ii) PARP cleavage detected by western blot and (iii) DNA fragmentation determined by TUNEL assay (FIG. 3). We conducted the TUNEL assay in both attached and detached cells after various treatments. It is rather interesting to note that almost all the detached cells were TUNEL-positive as evaluated by both flow cytometry and confocal microscopy, while virtually there are no apoptotic cells in the attached cells (FIG. 3C). Therefore, the percentage of detached cells was subsequently used as an index for the quantitative measurement of apoptosis and the results are presented in FIG. 4. Parthenolide (25 μM) or TNF-α (15 ng/ml) treatment alone for 24 h does not cause any significant changes of cell morphology, PARP cleavage and cell detachment, as demonstrated in FIGS. 3A, 3B and 4B, respectively. Parthenolide pre-treatment enhanced TNF-α mediated apoptosis in a dose-dependent manner. At 25 μM, nearly 60% of the cells were detached apoptotic cells (FIG. 4). The IC₅₀ is calculated to be 23 μM. Such results were supported by the significant cell morphological changes and PARP cleavage (Panel d in FIGS. 3A and 3B). PDTC pre-treatment also enhanced apoptotic cell death in TNF-α treated cells, but to a much lesser extent than that of parthenolide. When cells were pretreated with Act D (a mRNA synthesis inhibitor) or CHX (a protein synthesis inhibitor) as positive controls, severe apoptosis was also observed. For instance, nearly 100% of cells were found to be detached apoptotic cells, accompanied by complete cleavage of PARP in Act D-pretreated cells. NS398 is a known specific COX-2 enzyme inhibitor (Hara et al., 1997; Liu et al., 1998). In the present study, we used a relatively low concentration of NS398 (10 μM) at which NS398 itself is not cytotoxic (data not shown). Similar to the effect of parthenolide, pre-treatment with NS398 also significantly enhanced TNF-α induced apoptosis (FIGS. 3A, 3B and 4B). Results from this part of our study thus suggest that COX-2 is one of molecular target of parthenolide in TNF-α stimulated CNE1 cells.

Parthenolide Inhibits TNF-α Induced NF-κB Activation in CNE1 Cells

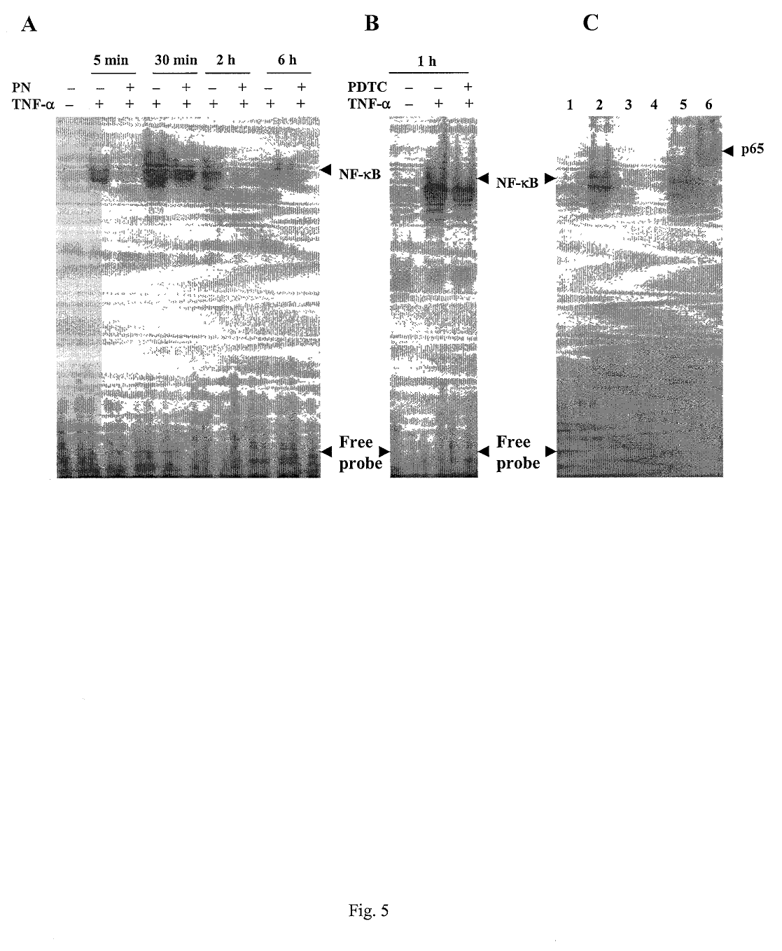

Earlier studies showed that parthenolide is capable of inhibiting NF-κB activation induced by a number of stimuli including phorbol 12-myristate 13-acetate (PMA), TNF-α, hydrogen peroxide and CD3/CD28 ligation in either Jurkat cells or HeLa cells (Bork et al., 1997; Hehner et al., 1998). In the present study we further investigated whether parthenolide is able to inhibit the NF-κB signalling pathway in TNF-α stimulated human NPC cells. TNF-α induced NF-κB activation in CNE1 cells were studied by (i) NF-κB DNA binding activity, (ii) IκBα degradation, (iii) p65 nuclear translocation, and (iv) NF-κB dependent gene transcription. As shown in FIG. 5A, parthenolide significantly inhibited the DNA binding activity of NF-κB determined by EMSA. Such inhibition starts as early as 5 min after TNF-α exposure and appears to be persistent until 6 h. We also tested the effect of PDTC, a well-known NF-κB inhibitor in our system (Schreck et al., 1992; D'Acquisto et al., 2000). It is found that PDTC is not effective at earlier time points (5 and 30 min, data not shown) and a certain degree of reduction was seen only at 1 h after TNF-α exposure (FIG. 5B). The specificity of the EMSA used in our test was confirmed by the complete inhibition of NF-κB DNA binding by excess amount of unlabeled NF-κB cold probe (FIG. 5C, lane 4), while a similar amount of nonspecific cold probe (AP-1) failed to affect the binding activity (FIG. 5C, lane 5). Moreover, majority of the NF-κB is found to be p65 as shown by the supershift assay (FIG. 5C, lane 6).

Parthenolide Inhibits IκBα Degradation, p65 Nuclear Translocation and DNA Binding in a Dose-dependent Manner

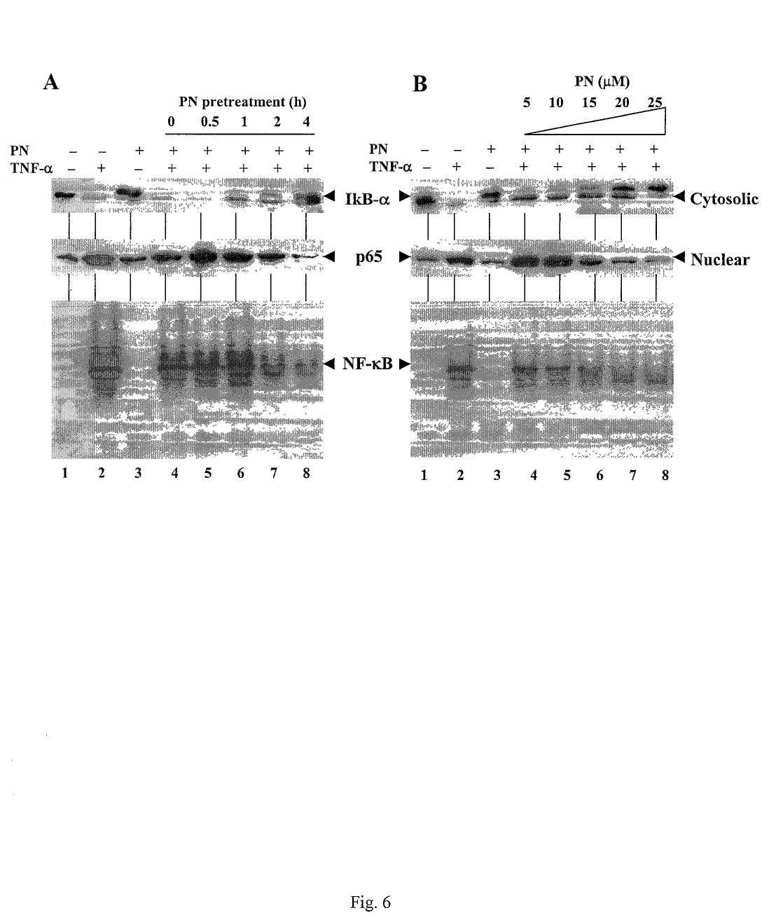

In this part of the experiment, we further studied effects of parthenolide on the sequential events including (i) cytoplasmic IκBα degradation, (ii) p65 nuclear translocation and (iii) NF-κB-DNA binding in TNF-α treated CNE1 cells. When cells were treated with TNF-α for 30 min, the unphosphorylated IκBα in the cytosolic fraction was completely degraded, accompanied by the significant increase of the amount of p65 and DNA binding activity in the nuclear, detected by western blot and EMSA, respectively (FIGS. 6A and 6B, lane 2). Parthenolide pre-treatment alone (25 μM×4 h) does not cause any of these changes (FIGS. 6A and 6B, lane 3). In order to optimise the parthenolide pre-treatment condition, CNE1 cells were first pretreated with parthenolide (25 μM) for a period ranging from 0 to 4 h. As shown in FIG. 6A, no protective effects were found when parthenolide was added with TNF-α simultaneously (lane 4) or with 0.5 h pre-treatment (lane 5). The protective effect was seen from 1 h onwards and 4 h pre-treatment offers the most significant inhibitory effect against IκBα degradation, p65 nuclear translocation and DNA binding (lane 8). Therefore, cells were pretreated with parthenolide for 4 h prior to TNF-α exposure in the subsequent dose-response study with results summarized in FIG. 6B. No evident inhibitory effects were found in the two lower doses (5 and 10 μM) (FIG. 6B, lanes 4 and 5), while higher concentrations of parthenolide (from 15 to 25 μM) significantly suppressed TNF-α induced IκBα degradation, NF-κB nuclear translocation and DNA binding in a dose-dependent manner (FIG. 6B, lanes 6, 7 and 8). Parthenolide has been found to act on the upstream kinases of IκB (IKK complex) to inhibit NF-κB activation (Hehner et al., 1999). Although the direct effect of parthenolide on IKK is not examined in the present study, the dose-dependent inhibition of parthenolide on IκBα degradation suggests that parthenolide may also act through a similar pattern to suppress the phosphorylation, ubiquitination and degradation of this inhibitor, which eventually prevents NF-κB activation in CNE1 cells.

Parthenolide Inhibits IκBα Nuclear Localization Induced by TNF-α in CNE1 Cells

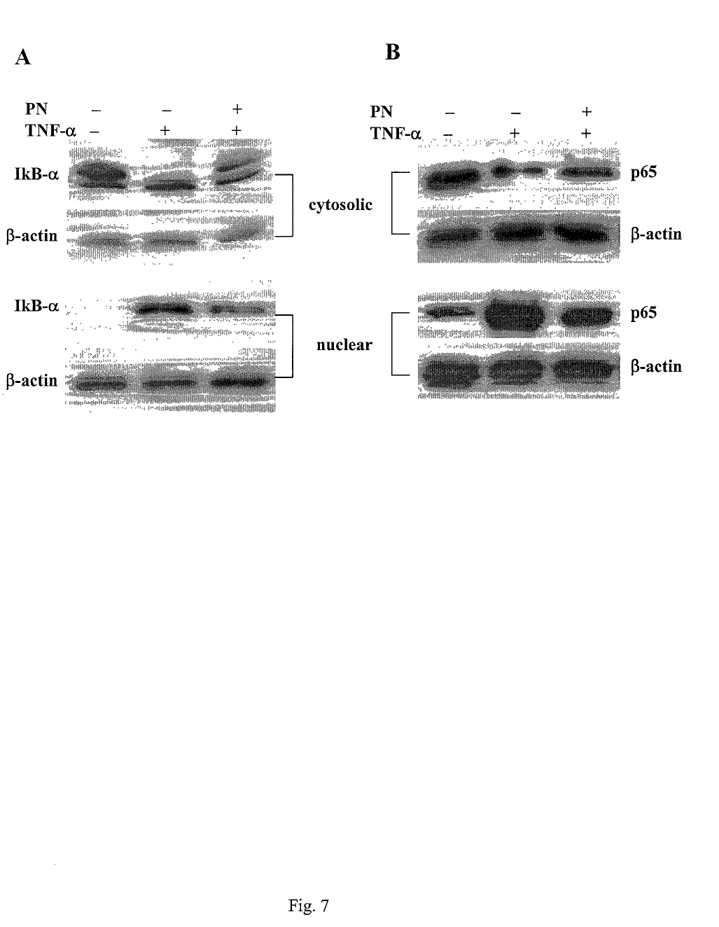

Apart from the well-known inhibitory function of IκBα on NF-κB in cytoplasm, recent studies have suggested that nuclear localization of IκBα is part of the physiological mechanism regulating NF-κB dependent transcription (Renard et al., 2000; Tran et al., 1997). Here we tested whether parthenolide would affect such a mechanism in TNF-α treated cells. No IκBα was detected in the nucleus of resting control cells, while there is a significant increase of IκBα content in nucleus 30 min after TNF-α exposure, concomitantly with the degradation of IκBα in the cytoplasm (FIG. 7A). Pretreatment with parthenolide (25 μM×4 h) tends to inhibit this process, although not completely, demonstrated by (i) increase of IκBα in the cytosolic fraction and (ii) decrease of this protein in the nucleus when compared to cells treated with TNF-α only (FIG. 7A). In parallel, the changes of p65 content in both cytosolic and nuclear fractions were also determined in the same cells. As shown in FIG. 7B, the significant increase of nuclear p65 well corresponded to the decrease of this NF-κB subunit in cytoplasm 30 min after TNF-α exposure. Similarly parthenolide treatment significantly reduced the nuclear content of p65 and increased its level in cytoplasm. Therefore, it seems that the nuclear localization of IκBα and p65 happens simultaneously after TNF-α stimulation and parthenolide is capable of inhibiting both processes.

Parthenolide Prevents the Transcriptional Activity of NF-κB

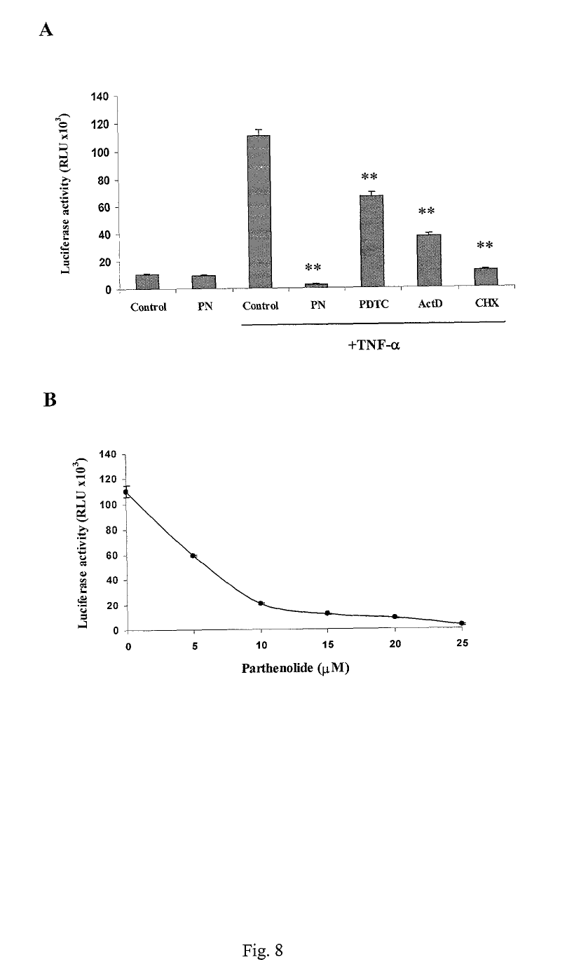

So far we have shown that parthenolide is capable of inhibiting IκBα degradation, p65 nuclear translocation and NF-κB-DNA binding. As DNA binding alone does not always correlate with NF-κB-dependent gene transcription, we further tested the inhibitory effects of parthenolide on the transcriptional activity of NF-κB, using a luciferase reporter gene assay. CNE1 cells were transiently transfected with either pNF-κB -luc vector or a control vector (pTAL-luc) and then stimulated with TNF-α in the presence of parthenolide and some other inhibitory agents. As demonstrated in FIG. 8A, TNF-α exposure increased the luciferase activity more than 10 times. While parthenolide treatment alone did not cause any detectable changes, the presence of parthenolide (25 μM) completely prevented TNF-α induced luciferase activation. Other inhibitors including PDTC, Act D and CHX all are capable of inhibiting NF-κB dependent transcription with an order of PDTC <Act D <CHX. Moreover, the inhibitory effect of parthenolide is also found to be dose-dependent with an IC₅₀ calculated to be 5 μM, indicating the high sensitivity of this test.

Direct Interference by Parthenolide with the DNA Binding Activity of NF-κB

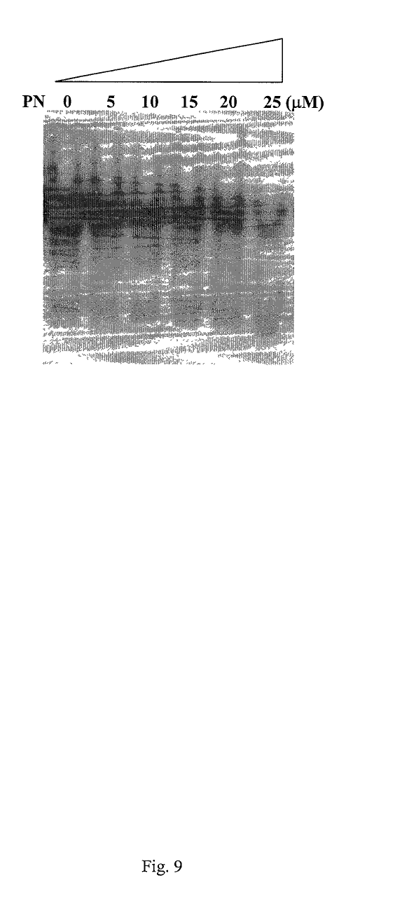

In this study, we noticed that the low concentrations of parthenolide (5 and 10 μM) significantly reduced the luciferase activity (FIG. 8B), which is different from the dose-response pattern of IκBα degradation and p65 nuclear translocation (FIG. 6B). Such discrepancy suggests that parthenolide may involve some other mechanisms to inhibit NF-κB dependent gene transcription. We thus investigated whether parthenolide is capable of directly interfering the DNA binding activity of activated NF-κB in nucleus. The nuclear extract from TNF-α-treated cells containing activated NF-κB were pooled and incubated with various concentrations of parthenolide (0 to 25 μM) for 1 h at room temperature. The incubated extracts were then analysed by EMSA. Results from FIG. 9 clearly show that parthenolide directly interferes with the DNA binding activity of activated NF-κB in a dose-dependent manner, and such effect is visible even at the two low concentrations of parthenolide (5 and 10 μM). Similar results were also observed when the cytosolic extracts from control cells containing resting NF-κB were treated with deoxycholate (0.8%×15 min) to free NF-κB from IκB binding, then treated with parthenolide (Manna and Aggarwal, 2000). Results from this part of our study are apparently inconsistent with an earlier report that parthenolide does not directly affect the DNA binding activity of activated NF-κB (Hehner et al., 1998).

Parthenolide Inhibits COX-2 Expression in TNF-α Treated CNE1 Cells

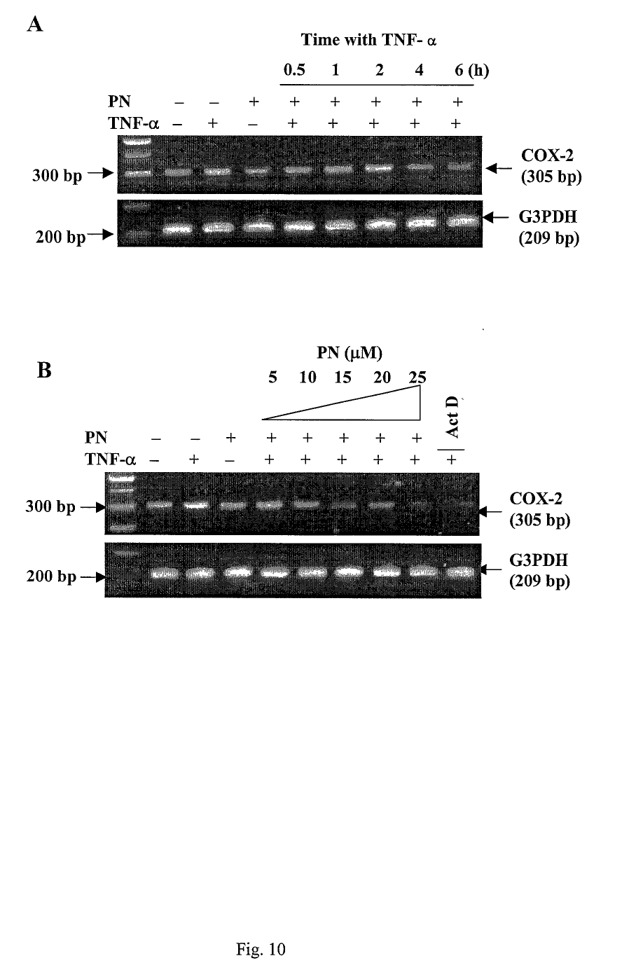

COX-2 is believed to be one of the target genes regulated by NF-κB, based on the fact that one or two putative NF-κB consensus sequences have been identified in the promoter region of human or mouse COX-2 gene acting as a positive regulatory element (Appleby et al., 1994; Tazawa et al., 1994; Yamamoto et al., 1995). An earlier study has suggested that parthenolide is able to inhibit COX-2 expression in lipopolysaccharide (LPS)-stimulated inflammatory cells (macrophages) (Hwang et al., 1996). However, currently it is not known whether parthenolide is capable of inhibiting COX-2 expression in cancer cells, following the suppression of NF-κB activation. Therefore, this part of the study was designed to examine the inhibitory effect of parthenolide on COX-2 expression by the determination of both COX-2 mRNA and protein level in TNF-α treated CNE1 cells. First, the mRNA level of COX-2 was determined using RT-PCR. As shown in FIG. 10, there is substantial amount of COX-2 mRNA in the unstimulated control cells, corresponding to the basal level of COX-2 protein in the control cells as shown earlier (FIG. 2A), while TNF-α exposure significantly enhanced COX-2 mRNA level (FIGS. 10A and 10B). The inhibitory effect of parthenolide pre-treatment (25 μM×4 h) on TNF-α induced COX-2 mRNA upregulation is found to be time-dependent. Evident reduction of COX-2 mRNA level was only observed after TNF-α exposure for more than 4 h in the presence of parthenolide (FIG. 10A). Moreover, parthenolide is capable of inhibiting COX-2 expression in a dose-dependent manner. All concentrations of parthenolide used in this study effectively reduced the mRNA level. With the highest concentration (25 μM), parthenolide almost completely inhibited the COX-2 mRNA transcription, well corresponding to the results of NF-κB transcription activity determined by luciferase reporter gene assay (FIG. 8). Act D was used a positive control which also markedly reduced the mRNA level in CNE1 cells (FIG. 10B).

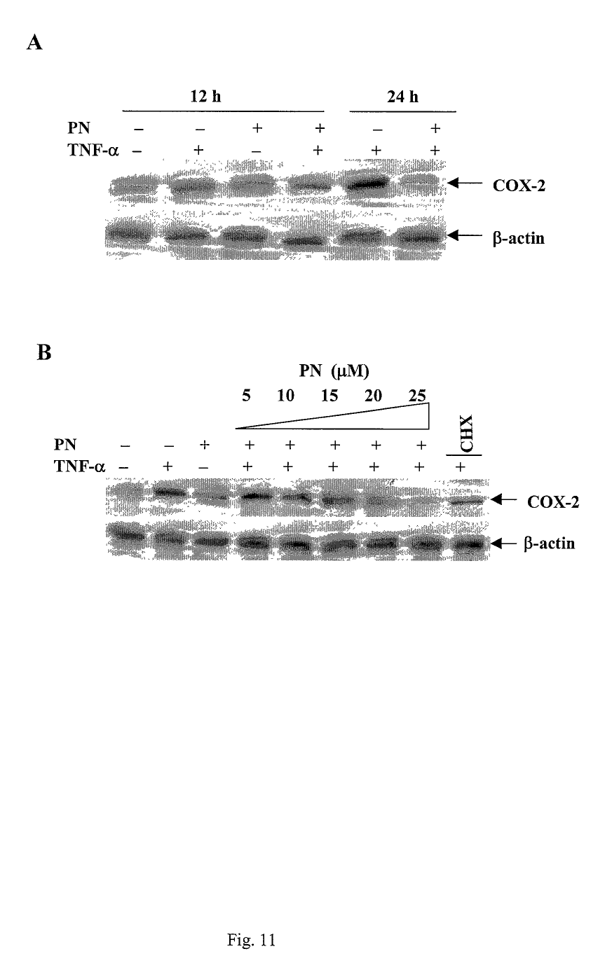

Following the determination of COX-2 mRNA transcription, we further measured the COX-2 protein level using western blot, and the results are summarized in FIG. 11. Upregulation of COX-2 protein level by TNF-α exposure was seen after cells were treated for more than 12 h and parthenolide pre-treatment (25 μM×4 h) is able to inhibit the COX-2 translation from 12 h onwards, with more evident effect at 24 h (FIG. 11A). We thus used this time point for the dose-response study as shown in FIG. 11B. All concentrations of parthenolide except the lowest one are capable of reducing the COX-2 protein level in CNE1 cells. Significant reduction was also observed in cells pretreated with CHX, a protein synthesis inhibitor used as a positive control.

Parthenolide Inhibits PGE₂ Production in CNE1 Cells

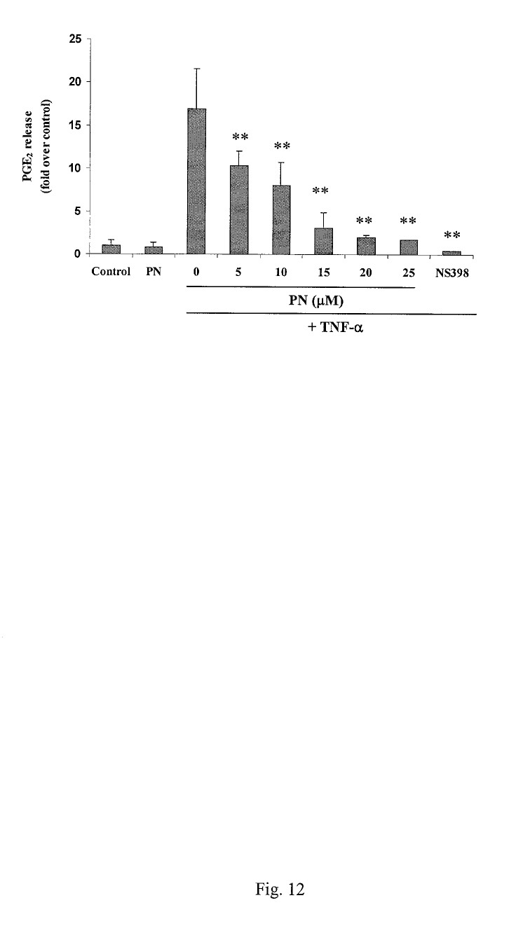

As described above, parthenolide dose-dependently inhibits COX-2 transcription and translation. In this part of the experiments, we further determined whether parthenolide inhibits PGE₂ production in TNF-α stimulated CNE1 cells. As shown in FIG. 12, TNF-α treatment for 24 h enhanced the PGE₂ level for more than 15 times compared to that in the control cells, well corresponding to the significant upregulation of both COX-2 mRNA and protein level (FIGS 9 and 10). Parthenolide alone does not alter the PGE₂ level significantly, while parthenolide pre-treatment suppressed TNF-α promoted PGE₂ production in a dose-dependent manner with an IC₅₀ around 10 μM. The two higher concentration groups almost completely inhibited the PGE₂ production, consistent with the changes of both COX-2 mRNA and protein levels as shown earlier (FIGS. 10 and 11). Meanwhile, NS398, a specific COX-2 enzyme inhibitor, completely abolished the PGE₂ production in TNF-α treated CNE1 cells.

Parthenolide has been found to react directly with COX enzyme to inhibit PGE₂ production in a cell-free system (Pugh and Sambo, 1988). In this study we also tested such an effect of parthenolide. CNE1 cells were first treated with TNF-α for 12 h, followed by parthenolide exposure for another 12 h. It was found that such post-treatment with parthenolide only marginally reduced the PGE₂ level by about 15% (p<0.05, data not shown). Therefore, it is believed that the direct modification of COX-2 enzyme activity by parthenolide is rather minimum in this study.

Human Colorectal Cancer Cells with Higher Level of COX-2 are More Susceptible to Parthenolide Cytotoxicity

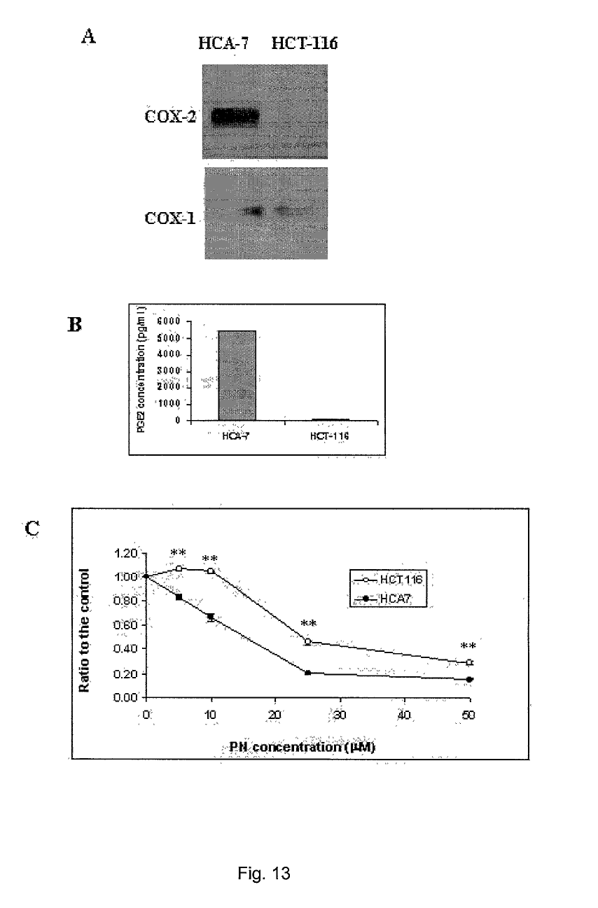

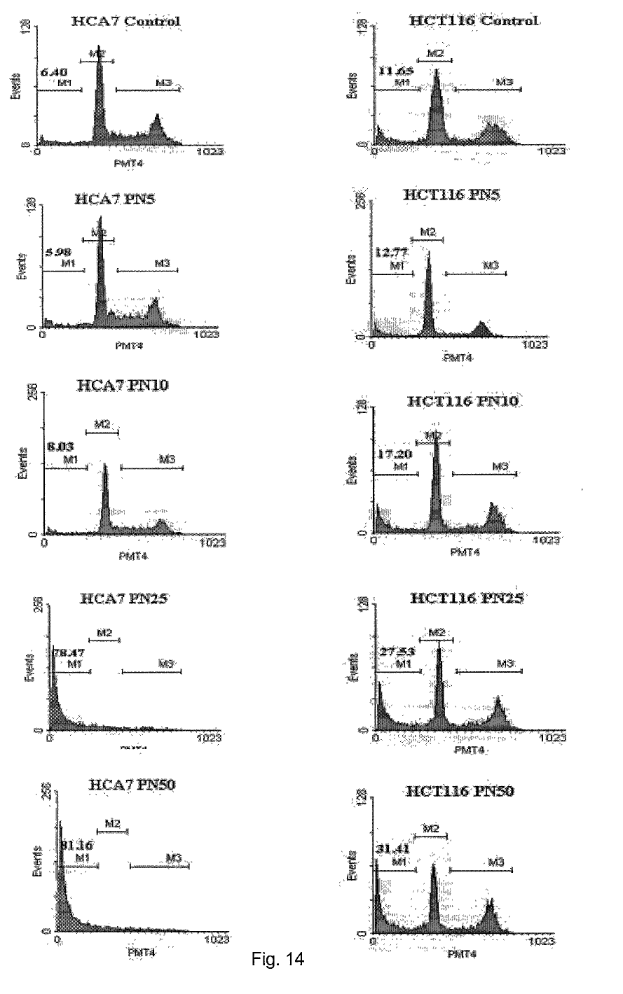

The basal levels of COX-2 and COX-1 were determined and the results are presented in FIG. 13A. HCA-7 cells possess a substantial amount of COX-2 protein while the COX-2 protein is not expressed in HCT-116 cells. In contrast, both cells contain a similar low level of COX-1. Such findings are consistent with earlier reports on the characteristics of these two cell lines (Brattain et al., 1981; Lampert et al., 1985; Sheng et al., 1997). Similarly, HCA-7 cells produce a much higher amount of PGE₂ than HCT-116 cells (FIG. 13B). More interestingly, it is noted that HCA-7 cells are much more susceptible to parthenolide toxicity, as determined by MTT assay (FIG. 13C) and the percentage of sub-G1 cells (FIG. 14). Such results are consistent with the findings that parthenolide selectively kills human NPC cells CNE1 with higher COX-2 levels, while spares CNE2 cells with low COX2 expression levels. Once again, the close association between the COX-2 expression and parthenolide cytotoxicity indicates that COX-2 might be one of the molecular targets for the cytotoxic effect of parthenolide.

Parthenolide Inhibits HCA-7 Cell Growth in vivo

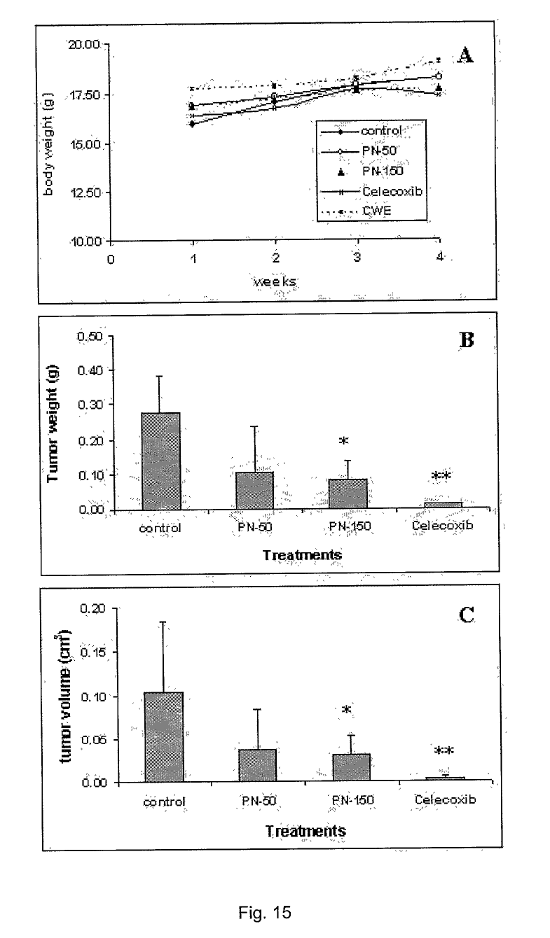

In order to further characterize the anticancer effect of parthenolide, a nude mice xenograft animal model was used. In the control group, the tumor slump became visible one week after HCA-7 cells were injected subcutaneously. At the time of sacrifice, the tumor was isolated and the size and weight was measured. In the trial experiment, it has been estimated that each animal each day consumes about 3 g of food and drinks about 3 ml of water and the maximal tolerated dose (MTD) of parthenolide is about 500 mg/kg body wt/day. As shown in FIG. 15A, the body weight of animals with various treatments did not change significantly from that of the control group, indicating that the doses of parthenolide or Celecoxib used in the present study were not toxic to the animals.

The inhibitory effect of parthenolide on cell growth was demonstrated by the significantly reduced tumor weight and size, as shown in FIGS. 15B and 15C, respectively. The high parthenolide dosage (150 mg/kg body wt/day) significantly reduced the tumor weight and volume as compared with the control group. Reduced tumor weight and volume was also observed in animals administered with low parthenolide dosage (50 mg/kg body wt/day), although no statistical significance was found. Celecoxib, as the positive control, almost completely blocked the tumor growth.

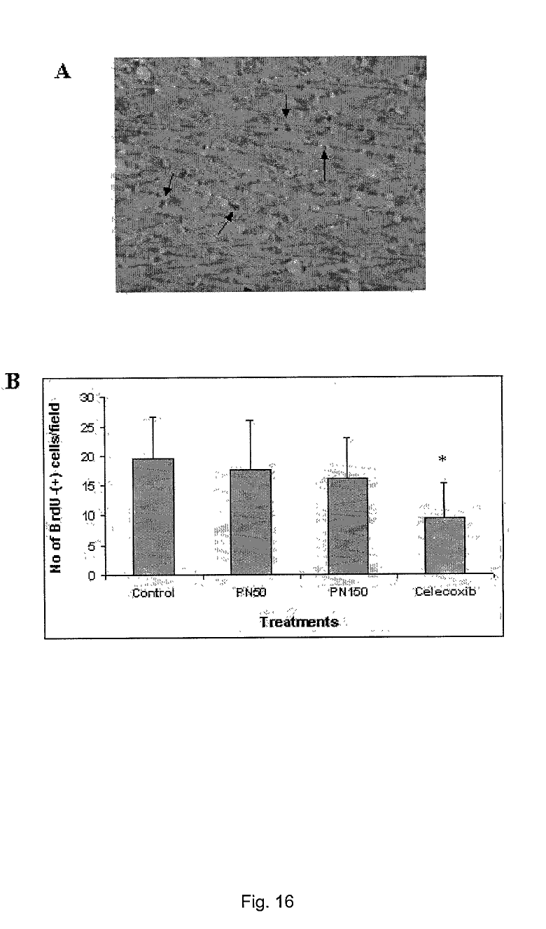

The above finding was generally supported by the results from the BrdU incorporation test. As shown in FIG. 16, parthenolide administration tends to decrease the rate of BrdU incorporation although no statistical significance was found compared to the control group. Celecoxib markedly reduced the percentage of BrdU-positive cells, indicating their significant inhibitory effect on tumor cell growth in vivo.

Parthenolide Induces Apoptotic Cell Death in HCA-7 Cells in vivo

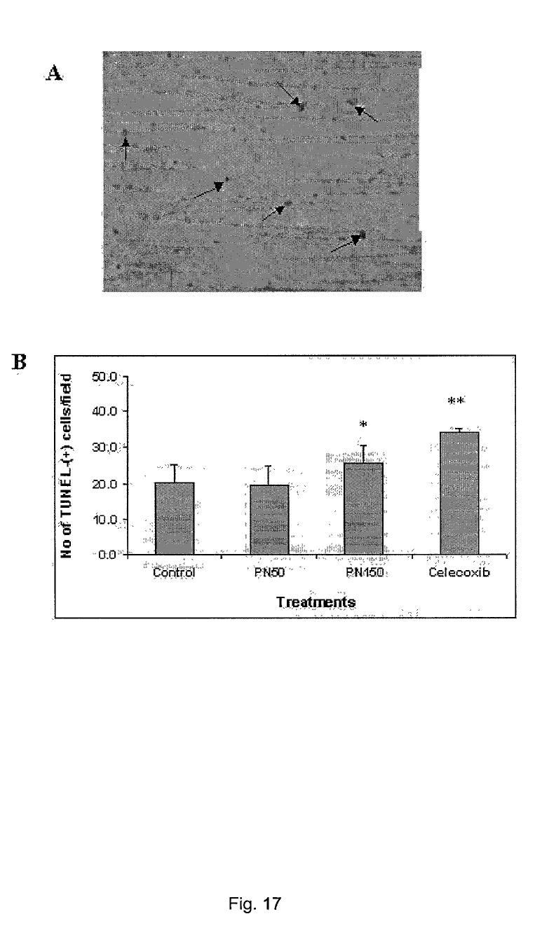

In the present study, parthenolide-induced apoptotic cell death in HCA-7 xenografts was examined using TUNEL assay and the results are presented in FIG. 17. The higher concentration of parthenolide (150 mg/kg body wt/day) significantly enhanced the percentage of TUNEL-positive cells, suggesting that parthenolide is capable of inducing apoptotic cell death under in vivo condition. Such a finding well explains the observation that parthenolide significantly reduced the xenografted tumor weight and size. Consistently, markedly increased apoptosis was also observed in Celecoxib treated mice.

### Example 4

### Discussion

The present invention for the first time establishes parthenolide-mediated apoptosis in cancer cells and that parthenolide, and chrysanthemum ethanolic extract which has as its major component parthenolide may therefore be used to prevent or treat cancer.

The inventors have attempted to elucidate the molecular mechanisms involved in parthenolide-mediated apoptosis in a human NPC cell line (CNE1 cells) and provided experimental evidence showing that COX-2 is one of main molecular targets of parthenolide: (i) cells with higher level of COX-2 are more susceptible to the cytotoxic effect of parthenolide (FIG. 2); (ii) pretreatment with non-toxic concentrations of parthenolide (<=25 μM) greatly sensitizes TNF-a-treated cells to apoptosis, an effect similar to that of NS398, a specific inhibitors of COX-2 enzyme activity (FIGS. 3 and 4); and (iii) parthenolide dose-dependently inhibits COX-2 expression and PGE₂ production stimulated by TNF-α in CNE1 cells (FIGS. 10 to 12). Many COX-2 inhibitors such as NSAIDs are capable of inhibiting cell growth and inducing apoptotic cell death in various cancer cells, mainly by their direct effect on COX-2 enzyme activity (Sheng et al., 1997; Chinery et al., 1998; Grossman et al., 2000; Rahman et al., 2000). In the present study, it is believed that parthenolide primarily act through the inhibition of COX-2 expression, as shown by the sequential reduction of COX-2 mRNA and protein level prior to the reduction of PGE₂ production, and the similar doseresponse pattern of these events. Based on the observation that parthenolide alone did not cause any reduction of PGE₂ level and that the post-treatment of parthenolide only marginally affected the PGE₂ level caused by TNF-α, it is believed that the direct interference of COX-2 enzyme activity by parthenolide is rather minimum, although such an effect has been observed in cell-free systems (Pugh and Sambo, 1988). Therefore, results from the present study for the first time demonstrate that parthenolide induced apoptotic cell death in TNF-α treated human NPC cells is mediated by the inhibition of COX-2 expression in human NPC cells.

The present invention also shows that parthenolide inhibits COX-2 expression through the NF-κB pathway. It is well known that COX-2 is one of the target genes regulated by NF-κB. The promoter region of human or mouse COX-2 gene has been cloned and one or two putative NF-κB consensus sequences were found as a positive regulatory element (Appleby et al., 1994; Tazawa et al., 1994; Yamamoto et al., 1995). Various NF-κB positive stimuli such as TNF-α and LPS are capable of activating NF-κB and promoting COX-2 expression in a temporal pattern (Schmedtje et al., 1997; Callejas et al., 1999; D'Acquisto et al., 2000). Moreover, NF-κB inhibitors such as PDTC or transfection with a super-repressor IκB inhibit NF-κB activation and suppress COX-2 expression accordingly (Jobin et al., 1998; Plummer et al., 1999; Kojima et al., 2000). In the present study, TNF-α exposure leads to a rapid activation of NF-κB, followed by the induction of COX-2 expression in a time and dose-dependent manner. Therefore, these results indicate that NF-κB activation is indeed responsible for the induction of COX-2 expression in TNF-α treated CNE1 cells. Parthenolide pre-treatment significantly reduced NF-κB activation in CNE1 cells, manifested by (i) inhibition of cytoplasmic IκBα degradation, (ii) decrease of p65 nuclear translocation, (iii) reduction of NF-κB DNA binding, and (iv) diminution of NF-κB dependent transcription, determined by western blot, EMSA and luciferase reporter gene assay, respectively. These findings are also consistent with previous studies showing that parthenolide is a potent NF-κB inhibitor in a number of cells including Jurkat cells, Hela cells and L929 fibroblasts stimulated with TNF-α, PMA, H₂O₂ or CD3/CD28 ligation (Bork et al., 1997; Hehner et al., 1998).

Such inhibitory effects are believed to be specific as parthenolide does not affect activities of other transcription factors such as AP-1 and Oct-1 (Bork et al., 1997; Hehner et al., 1998). The main reason that explains such specificity of parthenolide on NF-κB is based on the observation that parthenolide targets IKK, the upstream kinase of IKB proteins which is the point of convergence for most NF-κB activating stimuli (Hehner et al., 1999; Karin and Ben-Neriah, 2000). Therefore, the close correlation between the inhibition of NF-κB activation and COX-2 expression by parthenolide in TNF-α stimulated CNE1 cells suggests such a mechanistic pathway: parthenolide first inhibits the activity of IKK, as a result, suppresses cytoplasmic IκBα phosphorylation and degradation, subsequently reduces p65 nuclear translocation, and eventually prevents NF-κB DNA binding and transcription. Such a mechanism is apparently supported by a recent observation that parthenolide inhibits iNOS expression through a similar NF-κB-dependent pathway in rat arotic smooth muscle cells stimulated with LPS (Wong and Menendez 1999).

Further, incubation of nuclear extracts containing activated NF-κB protein with parthenolide led to significant reduction of its DNA-binding activity (FIG. 9). It is thus suggested parthenolide may also directly interfere with NF-κB and DNA binding through the reaction of its active site with the sulfhydryl group of cysteine residues in the DNA binding domain of NF-κB. Although contradictory results were found in an earlier study (Hehner et al., 1998), the above finding well explains the observation that significant reduction of DNA binding activity and luciferase transcription in the two lower parthenolide concentration groups (5 and 10 μM) (FIGS. 6 and 8), without evident effect on IκBα degradation and p65 nuclear translocation (FIG. 6).

In addition to the detection of cytoplasmic IκBα degradation occurring after TNF-α treatment, we also determined the effect of parthenolide pre-treatment on nuclear localization of IκBα, a process which has been considered as part of the physiological mechanism regulating NF-κB dependent transcription (Renard et al., 2000; Tran et al., 1997). Based on the belief that nuclear IκBα inhibits NF-κB activation by preventing NF-κB-DNA binding and dissociating combined NF-κB from specific DNA consensus sequences (Zabel and Baeuerle, 1990; Tran et al., 1997), it was expected to see parthenolide would strengthen such a process. To our surprise, however, parthenolide pre-treatment significantly reduced the nuclear content of IκBα in TNF-α treated cells, concomitantly with the reduction of p65 level in the nucleus (FIG. 5). It seems that nuclear IκBα is not directly involved in the mechanism leading to the inhibition of NF-κB activation by parthenolide, but rather, the reduced IκBα results from reduced availability of free IκBα in the cytoplasm due to enhanced IκBα degradation caused by parthenolide pre-treatment. Since parthenolide is known to specifically act on IKK, the upstream kinases of IκBα (Hehner et al., 1999), our results also suggest the participation of IKK in the process of IκBα nuclear localization.

NS398, a specific inhibitor of COX-2 displays a higher potency for the inhibition of PGE₂ production than parthenolide, but it is less effective in apoptosis induction. The results from the present study therefore indicate that parthenolide may affect anti-apoptotic genes controlled by NF-κB other than COX-2 and that parthenolide may be used to treat or prevent cancer other than cancer with increased expression of COX-2.

Prior to this study, the anti-carcinogenic property of SL including parthenolide was poorly understood. Accumulating evidence from epidemiological investigations, clinical trials, animal models, and various in vitro experiments supports the critical role of COX-2 in a number of human cancers (Prescott and Fitzpatrick, 2000; Williams et al., 1999; Dubois et al., 1998; Taketo, 1998a; 1998b). In recent years, there is enormous interest surrounding dietary approaches towards cancer prevention. Both NF-κB and COX-2 become increasingly important targets for the identification of such dietary components. For instance, dietary flavone, the core structure of dietary flavonoids, is a potent apoptosis inducer in human colon cancer cells, a process closely associated with changed expression of COX-2 and NF-κB (Wenzel et al., 2000). trans-Resveratrol, a natural phytoalexin from grapes with well known anticancer property (Jang et al., 1997; Jang and Pezzuto, 1999), has also been found to act through a similar mechanism (Subbaramaiah et al., 1998; Holmes-McNary and Baldwin, 2000). In the present study, parthenolide is found to have a similar potency to the abovementioned compounds with a IC₅₀ around 23 μM. The above finding that parthenolide, as an active ingredient of herbs with anti-inflammatory properties, is capable of increasing the sensitivity of cancer cells to apoptotic cell death through the inhibitory effect on NF-κB mediated COX-2 expression is significant because (i) parthenolide can be used as a direct chemopreventive or chemotherapeutical agent in cancers with an increased level of COX-2 or with an increased constitutive activation of NF-κB; and (ii) parthenolide can be used in combination with and to complement other chemotherapy, including with other apoptotic drugs or to enhance the efficacy of cancer drugs or treatment known to trigger activation of NF-κB.

The presence and regulation of COX isoforms have not been previously documented in human NPC cells. Our results clearly demonstrate that the two isoforms, COX-1 and COX-2, are present in two most commonly used human NPC cell lines (CNE1 and CNE2), while unstimulated CNE1 cells appear to have a high level of COX-2 protein compared to CNE2 cells. Therefore, results from the present study indicate that an increased expression of COX-2 may play an important role in certain types of human NPC and that parthenolide can be administered as an anti-cancer agent to control or prevent this rather common cancer in certain regions of Asia.

Parthenolide may also be used to treat or prevent other common cancers with an increased expression of COX-2 expression, such as colorectal cancer. The anticancer property of parthenolide was evaluated in two human colorectal cancer cell lines (HCA-7 and HCT-116 cells) in two test systems (in vitro cell culture and in vivo nude mice xenograft model). Both cell lines have been well characterized and widely used in colorectal cancer study (Brattain et al., 1981; Lampert et al., 1985; Sheng et al., 1997). HCA-7 cells with high COX-2 expression and PGE₂ production are much more susceptible to parthenolide-induced cell growth inhibition and apoptosis, while HCT-116 with no COX-2 expression and low PGE₂ production are less sensitive, similar to the differential effect of parthenolide on CNE1 and CNE2 cells.

The nude mice-xenograft model has been well established for evaluation of anti-tumor agents (Sharkey and Fogh, 1984; Mattem et al., 1988). For instance, the anti-cancer effect of nonsteroidal anti-inflammatory drugs has been studied extensively using this test system (Sheng et al., 1997; Goldman et al., 1998; Goluboffet al., 1999; Sawaoka et al., 1998; 1999). At a non-toxic concentration, parthenolide dose-dependently inhibits HCA-7 cell proliferation in vivo, as evidenced by (i) reduced tumor weight and volume, (ii) decreased rate of BrdU incorporation, and (iii) enhanced apoptotic cell death. Celecoxib, a known selective COX-2 inhibitor (Williams et al., 2000), was used as the positive control and strong inhibitory effect was observed, confirming the validity of this test system. Parthenolide is therefore capable of inhibiting human cancer cell growth in cell culture in vitro and in vivo.

Currently there is substantial evidence that COX-2 plays a critical role in the tumorigenesis of various types of cancers including colorectal cancer, prostate cancer, breast cancer, skin cancer, etc. (Prescott and Fitzpatrick, 2000; Dubois et al., 1998). It is also known that COX-2 is one of the anti-apoptotic genes under the regulation of NF-κB and is capable of promoting cell growth and proliferation (Sellers and Fisher, 1999; Prescott and Fitzpatrick, 2000). Therefore, COX-2 becomes an important molecular target for cancer prevention and treatment and the inventors have shown that COX-2 is a molecular target of parthenolide in treating and preventing cancer.

### REFERENCES

Aggarwal, B. B. (2000) Apoptosis and nuclear factor-kappab: a tale of association and dissociation. Biochem. Pharmacol. 60, 1033-1039

Appleby, S. B., Ristimaki, A., Neilson, K., Narko, K., and Hla, T. (1994) Structure of the human cyclo-oxygenase-2 gene. Biochem. J. 302, 723-727

Baichwal, V. R., and Baeuerle, P. A. (1997) Activate NF-kappa B or die? Curr. Biol. 7, R94-96

Barkett, M., and Gilmore, T. D. (1999) Control of apoptosis by Rel/NF-kappaB transcription factors. Oncogene 18, 6910-24

Berry, M. I. (1984) Feverfew faces the future. Pharm. J. 232, 611-614

Bork, P. M., Schmitz, M. L., Kuhnt, M., Escher, C., and Heinrich, M. (1997) Sesquiterpene lactone containing Mexican Indian medicinal plants and pure sesquiterpene lactones as potent inhibitors of transcription factor NF-kappaB. FEBS Lett. 402, 85-90

Brattain, M. G., Fine, W. D., Khaled, F. M., Thompson, J., and Brattain D E. (1981) Heterogeneity of malignant cells from a human colonic carcinoma. Cancer Res. 41:1751-1756.

Callejas, N. A., Casado, M., Bosca, L., and Martin-Sanz, P. (1999) Requirement of nuclear factor kappaB for the constitutive expression of nitric oxide synthase-2 and cyclooxygenase-2 in rat trophoblasts. J Cell Sci. 18, 3147-3155

Cao, Y., Pearman, A. T., Zimmerman, G. A., McIntyre, T. M., and Prescott, S. M. (2000) Intracellular unesterified arachidonic acid signals apoptosis. Proc. Natl. Acad. Sci. U.S. A. 97, 11280-11285

Capasso, F. (1986) The effect of an aqueous extract of Tanacetum parthenium L. on arachidonic acid metabolism by rat peritoneal leucocytes. J Pharm Pharmacol. 38:71-72.

Chan, T. A., Morin, P. J., Vogelstein, B., and Kinzler, K. W. (1998) Mechanisms underlying nonsteroidal antiinflammatory drug-mediated apoptosis. Proc. Natl. Acad. Sci. U.S.A. 95, 681-686

Chen, F., Castranova, V., Shi, X., and Demers, L. M. (1999) New insights into the role of nuclear factor-kappaB, a ubiquitous transcription factor in the initiation of diseases. Clin. Chem. 45, 7-17

Chinery, R., Beauchamp, R. D., Shyr, Y., Kirkland, S. C., Coffey, R. J., and Morrow, J. D. (1998) Antioxidants reduce cyclooxygenase-2 expression, prostaglandin production, and proliferation in colorectal cancer cells. Cancer Res. 58, 2323-2327

D'acquisto, F., Lanzotti, V., and Carnuccio, R. (2000) Cyclolinteinone, a sesterterpene from sponge Cacospongia linteiformis, prevents inducible nitric oxide synthase and inducible cyclo-oxygenase protein expression by blocking nuclear factor-kappaB activation in J774 macrophages. Biochem. J. 346, 793-798

Dubois, R. N., Abramson, S. B., Crofford, L., Gupta, R. A., Simon, L. S., Van De Putte, L. B., and Lipsky, P. E. (1998) Cyclooxygenase in biology and disease. FASEB J. 12, 1063-1073

Gallois, C., Habib, A., Tao, J., Moulin, S., Maclouf, J., Mallat, A., and Lotersztajn, S. (1998) Role of NF-kappaB in the antiproliferative effect of endothelin-1 and tumor necrosis factor-alpha in human hepatic stellate cells. Involvement of cyclooxygenase-2. J. Biol. Chem. 273, 23183-23190

Goldman, A. P., Williams, C. S., Sheng, H., Lamps, L. W., Williams, V. P., Pairet, M., Morrow, J. D., and DuBois, R. N. (1998) Meloxicam inhibits the growth of colorectal cancer cells. Carcinogenesis 19:2195-2199.

Goluboff, E. T., Shabsigh, A., Saidi, J. A., Weinstein, I. B., Mitra, N., Heitjan, D., Piazza, G. A., Pamukcu, R., Buttyan, R., and Olsson, C. A. (1999) Exisulind (sulindac sulfone) suppresses growth of human prostate cancer in a nude mouse xenograft model by increasing apoptosis. Urology 53:440-445.

Gorczyca W, Gong J, Darzynkiewicz Z. (1993) Detection of DNA strand breaks in individual apoptotic cells by the in situ terminal deoxynucleotidyl transferase and nick translation assays. Cancer Res. 53:1945-1951.

Grossman, E. M., Longo, W. E., Panesar, N., Mazuski, J. E., and Kaminski, D. L. (2000) The role of cyclooxygenase enzymes in the growth of human gall bladder cancer cells. Carcinogenesis 21, 1403-1409

Grossmann, M., Nakamura, Y., Grumont, R., and Gerondakis, S. (1999) New insights into the roles of ReL/NF-kappa B transcription factors in immune function, hemopoiesis and human disease. Int. J. Biochem. Cell Biol. 31, 1209-1219

Hall, I. H., Williams, W. L. J., Grippo, A. A., Lee, K. H., Holbrook, D. J., and Chaney, S. G. (1988) Inhibition of nucleic acid synthesis in P-388 lymphocytic leukemia cells in culture by sesquiterpene lactones. Anticancer Res. 8, 33-42

Hanif, R., Pittas, A., Feng, Y., Koutsos, M. I., Qiao, L., Staiano-Coico, L., Shiff, S. I., and Rigas, B. (1996) Effects of nonsteroidal anti-inflammatory drugs on proliferation and on induction of apoptosis in colon cancer cells by a prostaglandin-independent pathway. Biochem. Pharmacol. 52, 237-245

Hara, A., Yoshimi, N., Niwa, M., Ino, N., and Mori, H. (1997) Apoptosis induced by NS-398, a selective cyclooxygenase-2 inhibitor, in human colorectal cancer cell lines. Jpn. J. Cancer Res. 88, 600-604

Hehner, S. P., Heinrich, M., Bork, P. M., Vogt, M., Ratter, F., Lehmann, V., Schulze-Osthoff, K., Droge, W., and Schmitz, M. L. (1998) Sesquiterpene lactones specifically inhibit activation of NF-kappa B by preventing the degradation of I kappa B-alpha and I kappa B-beta. J. Biol. Chem. 273, 1288-1297

Hehner, S. P., Hofmann, T. G., Droge, W., and Schmitz, M. L. (1999) The antiinflammatory sesquiterpene lactone parthenolide inhibits NF-kappa B by targeting the I kappa B kinase complex. J. Immunol. 163, 5617-5623

Holmes-McNary, M., and Baldwin, A. S. Jr. (2000) Chemopreventive properties of trans-resveratrol are associated with inhibition of activation of the IkappaB kinase. Cancer Res. 60, 3477-3483

Hwang, D., Fischer, N. H., Jang, B. C., Tak, H., Kim, J. K., and Lee, W. (1996) Inhibition of the expression of inducible cyclooxygenase and proinflammatory cytokines by sesquiterpene lactones in macrophages correlates with the inhibition of MAP kinases. Biochem. Biophys. Res. Commun. 226, 810-818

Israel, A. (2000) The IKK complex: an integrator of all signals that activate NF-kappaB? Trends Cell Biol. 10, 129-133

Jang, M., and Pezzuto, J. M. (1999) Cancer chemopreventive activity of resveratrol. Drugs Exp. Clin. Res. 25, 65-77

Jang, M., Cai, L., Udeani, G. O., Slowing, K. V., Thomas, C. F., Beecher, C. W., Fong, H. H., Farnsworth, N. R., Kinghom, A. D., Mehta, R. G., Moon, R. C., and Pezzuto, J. M. (1997) Cancer chemopreventive activity of resveratrol, a natural product derived from grapes. Science 275, 218-220

Jobin, C., Morteau, O., Han, D. S., and Balfour Sartor R. (1998) Specific NF-kappaB blockade selectively inhibits tumour necrosis factor-alpha-induced COX-2 but not constitutive COX-1 gene expression in HT-29 cells. Immunology 95, 537-543

Karin, M. (1999) The beginning of the end: IkappaB kinase (IKK) and NF-kappaB activation. J. Biol. Chem. 274, 27339-27342

Karin, M., and Ben-Neriah, Y. (2000) Phosphorylation meets ubiquitination: the control of NF-[kappa]B activity. Annu. Rev. Immunol. 18, 621-663

Kojima, M., Morisaki, T., Izuhara, K., Uchiyama, A., Matsunari, Y., Katano, M., and Tanaka, M. (2000) Lipopolysaccharide increases cyclo-oxygenase-2 expression in a colon carcinoma cell line through nuclear factor-kappa B activation. Oncogene 19, 1225-1231

Kujubu, D. A., Fletcher, B. S., Varnum, B. C., Lim, R. W., and Herschman, H. R. (1991) TIS10, a phorbol ester tumor promoter-inducible mRNA from Swiss 3T3 cells, encodes a novel prostaglandin synthase/cyclooxygenase homologue. J. Biol. Chem. 266, 12866-12872

Kutchera, W., Jones, D. A., Matsunami, N., Groden, J., McIntyre, T. M., Zimmerman, G. A., White, R. L., and Prescott, S. M. (1996) Prostaglandin H synthase 2 is expressed abnormally in human colon cancer: evidence for a transcriptional effect. Proc. Natl. Acad. Sci. U. S. A. 93, 4816-4820

Lampert, I. A., Kirkland, S., Farrell, S., and Borysiewicz, L. K. (1985) HLA-DR expression in a human colonic carcinoma cell line. J. Pathol. 146:337-344.

Li, C. C., Yu, M. C., and Henderson, B. E. (1985) Some epidemiologic observations of nasopharyngeal carcinoma in Guangdong, People's Republic of China. Natl. Cancer Inst. Monogr. 69, 49-52.

Li, J. H., Li, P., Klamut, H., and Liu, F. F. (1997) Cytotoxic effects of Ad5CMV-p53 expression in two human nasopharyngeal carcinoma cell lines. Clin. Cancer Res. 3, 507-514

Liu, X. H., Yao, S., Kirschenbaum, A., and Levine, A. C. (1998) NS398, a selective cyclooxygenase-2 inhibitor, induces apoptosis and down-regulates bcl-2 expression in LNCaP cells. Cancer Res. 58, 4245-4249

Manna, S. K., and Aggarwal, B. B. (2000) Vesnarinone suppresses TNF-induced activation of NF-kappa B, c-Jun kinase, and apoptosis. J. Immunol. 164, 5815-5825

Mattern J, Bak M, Hahn EW, Volm M. (1988) Human tumor xenografts as model for drug testing. Cancer Metastasis Rev. 7:263-284.

Nicoletti I, Migliorati G, Pagliacci M C, Grignani F, Riccardi C. (1991) A rapid and simple method for measuring thymocyte apoptosis by propidium iodide staining and flow cytometry. J. Immunol. Methods 139:271-279.

Pahl, H. L. (1999) Activators and target genes of Rel/NF-kappaB transcription factors. Oncogene 18, 6853-6866

Patel, N. M., Nozaki, S., Shortle, N. H., Bhat-Nakshatri, P., Newton, T. R., Rice, S., Gelfanov, V., Boswell, S. H., Goulet, R. J. Jr., Sledge, G. W. Jr., and Nakshatri, H. (2000) Paclitaxel sensitivity of breast cancer cells with constitutively active NF-kappaB is enhanced by IkappaBalpha super-repressor and parthenolide. Oncogene 19, 4159-4169

Plummer, S. M., Holloway, K. A., Manson, M. M., Munks, R. J., Kaptein, A., Farrow, S., and Howells, L. (1999) Inhibition of cyclo-oxygenase 2 expression in colon cells by the chemopreventive agent curcumin involves inhibition of NF-kappaB activation via the NIK/IKK signalling complex. Oncogene 18, 6013-6020

Prescott, S. M., and Fitzpatrick, F. A. (2000) Cyclooxygenase-2 and carcinogenesis. Biochim. Biophys. Acta. 1470, M69-78

Pugh, W. J., and Sambo, K. (1988) Prostaglandin synthetase inhibitors in feverfew. J. Pharm. Pharmacol. 40, 743-745

Rahman, M. A., Dhar, D. K., Masunaga, R., Yamanoi, A., Kohno, H., and Nagasue, N. (2000) Sulindac and exisulind exhibit a significant antiproliferative effect and induce apoptosis in human epatocellular carcinoma cell lines. Cancer Res. 60, 2085-2089

Renard, P., Percherancier, Y., Kroll, M., Thomas, D., Virelizier, J. L., Arenzana-Seisdedos, F., and Bachelerie, F. (2000) Inducible NF-kappaB activation is permitted by simultaneous degradation of nuclear IkappaBalpha. J. Biol. Chem. 275, 15193-15199

Ross, J. J., Arnason, J. T., and Bimboim, H. C. (1999) Low concentrations of the feverfew component parthenolide inhibit in vitro growth of tumor lines in a cytostatic fashion. Planta. Med. 65, 126-129

Sano, H., Kawahito, Y., Wilder, R. L., Hashiramoto, A., Mukai, S., Asai, K., Kimura, S., Kato, H., Kondo, M., and Hla, T. (1995) Expression of cyclooxygenase-1 and -2 in human colorectal cancer. Cancer Res. 55, 3785-3789

Sawaoka, H., Kawanom, S., Tsuji, S., Tsujii, M., Gunawan, E. S., Takei, Y., Nagano, K., and Hori, M. (1998) Cyclooxygenase-2 inhibitors suppress the growth of gastric cancer xenografts via induction of apoptosis in nude mice. Am J Physiol. 274:G1061-1067.

Sawaoka, H., Tsuji, S., Tsujii, M., Gunawan, E. S., Sasaki, Y., Kawano, S., and Hori, M. (1999) Cyclooxygenase inhibitors suppress angiogenesis and reduce tumor growth in vivo. Lab Invest. 79:1469-1477.

Schmedtje, J. F. Jr., Ji, Y. S., Liu, W. L., DuBois, R. N., and Runge, M. S. (1997) Hypoxia induces cyclooxygenase-2 via the NF-kappaB p65 transcription factor in human vascular endothelial cells. J. Biol. Chem. 272, 601-608

Schreck, R., Meier, B., Mannel, D. N., Droge, W., Baeuerle, P. A. (1992) Dithiocarbamates as potent inhibitors of nuclear factor kappa B activation in intact cells. J. Exp. Med. 175, 1181-1194

Sellers, W. R. and Fisher, D. E. (1999) Apoptosis and cancer drug targeting. J. Clin Invest 104:1655-1661

Sharkey, F. E., and Fogh, J. (1984) Considerations in the use of nude mice for cancer research. Cancer Metastasis Rev. 3:341-360.

Shen, H. M., Ong, C. N., and Shi, C. Y. (1995) Involvement of reactive oxygen species in aflatoxin B1-induced cell injury in cultured rat hepatocytes. Toxicology 99, 115-123

Shen, H. M., Yang, C. F., Liu, J., and Ong, C. N. (2000) Dual role of glutathione in selenite-induced oxidative stress and apoptosis in human hepatoma cells. Free Radic Biol Med. 28:1115-1124.

Shen, H. M., Yang, C. F., Liu, J., Ding, W. X., and Ong, C. N. (2001) Oxidative stress mediated apoptotic signaling pathway in sodium selenite-treated HepG2 cells: mitochondria serve as the main target. Free Radic. Biol. Med. 30:9-21.

Sheng, H., Shab, J., Kirkland, S. C., Isakson, P., Coffey, R. J., Morrow, J., Beauchamp, R. D., and DuBois, R. N. (1997) Inhibition of human colon cancer cell growth by selective inhibition of cyclooxygenase-2. J. Clin. Invest. 99, 2254-2259

Sheng, H., Shao, J., Morrow, J. D., Beauchamp, R. D., and DuBois, R. N. (1998) Modulation of apoptosis and Bcl-2 expression by prostaglandin E2 in human colon cancer cells. Cancer Res. 58, 362-366

Shiff, S. J., Qiao, L., Tsai, L. L., and Rigas, B. (1995) Sulindac sulfide, an aspirin-like compound, inhibits proliferation, causes cell cycle quiescence, and induces apoptosis in HT-29 colon adenocarcinoma cells. J. Clin. Invest. 96, 491-503

Sizhong, Z., Xiukung, G., and Yi, Z. (1983) Cytogenetic studies on an epithelial cell line derived from poorly differentiated nasopharyngeal carcinoma. Int. J. Cancer 31, 587-590

Subbaramaiah, K., Chung, W. J., Michaluart, P., Telang, N., Tanabe, T., Inoue, H., Jang, M., Pezzuto, J. M., and Dannenberg, A. J. (1998) Resveratrol inhibits cyclooxygenase-2 transcription and activity in phorbol ester-treated human mammary epithelial cells. J. Biol. Chem. 273, 21875-21882

Sumner, H., Salan, U., Knight, D. W., Hoult, J. R. (1992) Inhibition of 5-lipoxygenase and cyclo-oxygenase in leukocytes by feverfew. Involvement of sesquiterpene lactones and other components. Biochem. Pharmacol. 43, 2313-2320

Taketo, M. M. (1998a) Cyclooxygenase-2 inhibitors in tumorigenesis (part I). J. Natl. Cancer Inst. 90, 1529-1536

Taketo, M. M. (1998b) Cyclooxygenase-2 inhibitors in tumorigenesis (Part II). J. Natl. Cancer Inst. 90, 1609-1620

Tazawa, R., Xu, X. M., Wu, K. K., and Wang, L. H. (1994) Characterization of the genomic structure, chromosomal location and promoter of human prostaglandin H synthase-2 gene. Biochem. Biophys. Res. Commun. 203, 190-199

Tran, K., Merika, M., and Thanos, D. (1997) Distinct functional properties of IkappaB alpha and IkappaB beta. Mol. Cell Biol. 17, 5386-5399

Wenzel, U., Kuntz, S., Brendel, M. D., and Daniel, H. (2000) Dietary flavone is a potent apoptosis inducer in human colon carcinoma cells. Cancer Res. 60, 3823-3831

Williams, C. S., Mann, M., and DuBois, R. N. (1999) The role of cyclooxygenases in inflammation, cancer, and development. Oncogene 18, 7908-7916

Williams, C. S., Watson, A. J., Sheng, H., Helou, R., Shao, J., and DuBois RN. (2000) Celecoxib prevents tumor growth in vivo without toxicity to normal gut: lack of correlation between in vitro and in vivo models. Cancer Res. 60:6045-6051.

Wong, H. R., and Menendez, I. Y. (1999) Sesquiterpene lactones inhibit inducible nitric oxide synthase gene expression in cultured rat aortic smooth muscle cells. Biochem. Biophys. Res. Commun. 262, 375-380

Woynarowski, J. M., and Konopa, J.(1981) Inhibition of DNA biosynthesis in HeLa cells by cytotoxic and antitumor sesquiterpene lactones. Mol Pharmacol. 19, 97-102

Yamamoto, K., Arakawa, T., Ueda, N., and Yamamoto, S. (1995) Transcriptional roles of nuclear factor kappa B and nuclear factor-interleukin-6 in the tumor necrosis factor alpha-dependent induction of cyclooxygenase-2 in MC3T3-E1 cells. J. Biol. Chem. 270, 31315-31320

Yang, C. F., Shen, H. M., and Ong, C. N. (1999) Protective effect of ebselen against hydrogen peroxide-induced cytotoxicity and DNA damage in HepG2 cells. Biochem. Pharmacol. 57:273-279.

Zabel, U., and Baeuerle, P. A. (1990) Purified human I kappa B can rapidly dissociate the complex of the NF-kappa Btranscription factor with its cognate DNA. Cell 61, 255-265.

We claim:

1. A method of preventing or treating cancer comprising administering to an animal in need of such prevention or treatment an effective amount of Chrysanthemum ethanolic extract.

2. The method according to claim 1 wherein the animal is a human patient.

3. The method according to claim 2 wherein the cancer is associated with an increased expression of COX-2.

4. The method according to claim 2 wherein the cancer is associated with an increased constitutive activation of NF-κB.

5. The method according to claim 2 wherein the cancer is colorectal cancer, nasopharyngeal cancer, prostate cancer, bladder cancer, brain tumour, breast cancer or skin cancer.

6. The method according to claim 5 wherein the extract is administered in combination with a pharmaceutically acceptable carrier or diluent.

7. The method according to claim 6 wherein the extract is administered orally.

8. A method of preventing or treating cancer comprising administering to an animal in need of such prevention or treatment an effective amount of parthenolide or derivative thereof.

9. The method according to claim 8 wherein the animal is a human patient.

10. The method according to claim 9 wherein the cancer is associated with an increased expression of COX-2.

11. The method according to claim 9 wherein the cancer is associated with an increased constitutive activation of NF-κB.

12. The method of according to claim 9 wherein the cancer is colorectal cancer, nasopharangeal cancer, prostate cancer, bladder cancer, brain tumour, breast cancer or skin cancer.

13. The method according to claim 12 wherein about 200 to 800 mg of parthenolide is administered daily in a single or multiple dose regimen.

14. The method according to claim 12 wherein parthenolide or derivative thereof is administered in combination with a pharmaceutically acceptable carrier or diluent.

15. The method according to claim 14 wherein parthenolide or derivative thereof is administered orally.

16. A kit comprising chrysanthemum ethanolic extract or parthenolide, or a derivative thereof and instructions for use in the treatment or prevention of cancer.

17. The kit according to claim 16 wherein the cancer is associated with an increased expression of COX-2.

18. The kit according to claim 16 wherein the cancer is associated with an increased constitutive activation of NF-κB.

19. The kit according to claim 16 wherein the cancer is colorectal cancer, nasopharangeal cancer, prostate cancer, bladder cancer, brain tumour, breast cancer or skin cancer.

20. A composition comprising parthenolide or derivative thereof and a pharmaceutically acceptable diluent or carrier.
